SEQUENCE LISTING (2 sequences)

SEQ ID NO: 1 - protein, 9 aa, artificial
  SA inhibitor with sequence derived from the SH3 binding domain of the human androgen receptor
  REPEAT (1)..(1): P can be repeated up to ten (n is 1 to 10)
  REPEAT (2)..(2): X can absent; X can be repeated (n is 0, 1 or 2)
PXHPHARIK

SEQ ID NO: 2 - protein, 10 aa, artificial
  SA inhibitor can be a peptide whose sequence is derived from the SH3 binding domain of the human androgen receptor
PPPHPHARIK

(19) United States
(12) Patent Application Publication
(10) Pub. No.: US 2023/0226138 A1
(43) Pub. Date: Jul. 20, 2023

(54) COMPOSITIONS AND METHODS FOR TREATING INFECTIONS AND NETOPATHY

(71) Applicant: Black Cat Bio Limited, Hinckley, Leicestershire (GB)

(72) Inventor: Mark Eccleston, Cambridgeshire (GB)

(73) Assignee: Black Cat Bio Limited, Hinckley, Leicestershire (GB)

(21) Appl. No.: 18/007,713

(22) PCT Filed: May 28, 2021
(86) PCT No.: PCT/EP2021/064324

§ 371 Date: Dec. 1, 2022

Related U.S. Application Data
(60) Provisional application No. 63/033,188, filed on Jun. 1, 2020.

(51) Int. Cl. A61K 38/08; A61K 31/455; A61K 38/46; A61K 31/706; A61P 37/06; A61K 9/00

(52) U.S. Cl. CPC A61K 38/08; A61K 31/455; A61K 38/465; A61K 31/706; A61P 37/06; A61K 9/0075

(57) ABSTRACT

The invention provides a pharmaceutical composition and methods for the treatment of a respiratory disease that results from a viral infection. The pharmaceutical composition can include a peptide that interferes with the Src family kinase-Androgen receptor interaction (i.e., an “SA inhibitor”). The composition can also include Niacin or a Niacin derivative and/or a DNase I or fragment or derivative. The pharmaceutical composition can prevent the development of Acute Respiratory Distress Syndrome (ARDS) associated with Corona vims infection and allow the host adaptive immune response to overcome the infection.

### CROSS-REFERENCE TO RELATED APPLICATIONS

The present application claims priority to U.S. Provisional Patent Application No. 63/033,188, filed Jun. 1, 2020, which is herein incorporated by reference in its entirety.

### FIELD OF INVENTION

The invention relates generally to pharmaceutical compositions, and more specifically, to a medical use of a pharmaceutical composition of one or more therapeutics to treat a respiratory disease caused by a viral infection.

### INTRODUCTION

The Coronavirus disease 2019 (COVID-19) outbreak in Wuhan, China started in the second half of 2019. It rapidly developed into a global pandemic in early 2020, resulting in the infection of millions of individuals and killing several hundred thousand individuals around the world in the first wave of infection. SARS CoV2 was identified as the infective agent. It has been seen that there are differences seen between individuals regarding the severity of disease. Many individuals display mild flu like symptoms, particularly in younger individuals. In contrast, older individuals, and particularly, those with underlying health conditions, may suffer severely from a COVID-19 infection. Among the debilitating results of the infection that have been seen are significant lung and peripheral organ damage with high rates of mortality. The term Severe Acute Respiratory Syndrome (SARS) has been used to refer to virally induced respiratory insult. There is currently no effective vaccination or treatment for SARS-CoV-2. Additionally, a gender bias has emerged where it is found that men develop more severe disease and having a higher level of mortality than women.

It has been hypothesized that androgen regulation via activation of two specific proteins involved in SARS CoV 2 infection may play a role in infectivity. Specifically, activation of the viral spike proteins by the transmembrane protease, serine 2 (TMPRSS2), may play a role in cell binding and is found on the surface of around 60% of lung alveolar cells (type 2 pneumonocytes) as well as other affected organs such as the kidney. ACE2, is the receptor for SARS-CoV 51. It is also the receptor for SARS CoV S2 and was previously reported to display androgen driven activity in prostate tissue. TMPRSS2 has been shown to proteolytically cleave ACE2 and increase infectivity of SARS-CoV 51. It has been hypothesized that it may play a similar role with SARS-CoV 2. Androgen driven susceptibility to COVID-19 severity may be exacerbated by genetic polymorphism, since both ACE2 and Androgen receptor genes are located on the male X chromosome. Additionally, several viruses, including coronaviruses, influenza viruses and flavivirus such as Dengue contain proline-rich, SH3 targeting motifs, similar to the Androgen receptor to hijack Src Kinase for replication (see, e.g. Pagano et al., Viral proteins and Src family kinases: Mechanisms of pathogenicity from a “liaison dangereuse”. World J Virol. 2013; 2(2):71-78. doi:10.5501/wjv.v2.i2.71Pagano MA. (2013)).

Additionally, Neutrophil Extracellular Traps (NETs) were first described as a specific innate immune response to systemic bacterial exposure in 2004. A hitherto unknown approach to pathogen response, NETosis, results in rapid externalisation of extracellular fibres made up of decondensed chromatin associated with the antimicrobial/granular protein components. The NETs can engulf and neutralise extracellular pathogens, including bacteria and fungi.

Recently, several viruses have been shown to initiate NET formation including influenza A, HIV-1, myxoma, encephalomyocarditis virus in humans, as well as feline Leukemia virus. Corona viruses such as SARS-CoV, MERS-CoV and COVID-19 also induce NET formation. Pathogen associated Molecular Patterns (PAMPs), essentially therapeutics from the virus, can stimulate NET release through a variety of mechanisms including, but not limited to, activation of TLR ligands, complement pathway and platelet binding. The activated neutrophils release NETs in a process referred to as NETosis, which can both immobilise viral particles via interaction with the DNA component, preventing dissemination and cellular uptake, as well as inactivating the virus by the action of the granule protein component including, but not limited to, MPO and defensins. It has been hypothesized that the primary rapid innate immune response and inflammatory regulatory effects of neutrophil activation and NETosis are critical in the hosts ability to fight off pathogens. However, sustained activation due to failure to clear pathogens is believed to result in host directed bystander effects and cause substantial collateral damage to local tissue. NETs can promote hypercoagulation and tissue disseminated thrombosis, which has been postulated as a final defence mechanism towards pathogen confinement in systemic sepsis.

Several pandemic viruses have demonstrated a propensity to induce Acute Respiratory Distress Syndrome (ARDS) through neutrophil recruitment to infected sites. Examples include Influenza, H1N1 (Avian Flu), SARS-CoV, MERS-CoV and SARS-CoV 2. While the pathophysiology of individual viruses may vary, a subset of those infected progress from mild to severe flu like symptoms leading to viral pneumonia that can further progress to ARDS and, in a limited set of individuals to multi-organ failure. ARDS in pathologic human Corona virus infection is believed to be associated with a catastrophic cytokine storm. The mechanism is not fully understood, but the cytokines regulate neutrophil activity and once released, it is believed that NETs can stimulate further cytokine production from macrophages which in turn stimulates further NETosis in the neutrophils generating a potential feedback loop and uncontrolled inflammation. Platelets may be involved in the activation process as they are associated with induction of NETs in Transfusion Related Acute Lung Injury (TRALI) which results in hypoxia and bilateral pulmonary edema within six hours of blood transfusion. These symptoms are also associated with SARS-CoV 2 disease severity.

These bystander effects can be directly mediated by enzymatic activity associated with components of the NETs, including myeloperoxidase and Neutrophil Elastase as noted in chronic kidney disease or toxicity of degradation products including free histones. Further, NET levels have been hypothesized to be associated with disease severity in SARS CoV 2. In addition to the acute effects of NET exposure, chronic exposure through recurrent inflammation can be highly deleterious. In many cases, the neutrophils from individuals with a disease appear to be predisposed to produce NETs either spontaneously or by stimulation.

Current vaccination methods for preventing SARS CoV2 have limitations and there is a need for therapeutics regardless of vaccines. Even with current vaccines, there is no guarantee that they provide long term immunity. Annual immunisation programs for seasonal flu have proved effective and are usually administered to “at risk” individuals ahead of the expected seasonal resurgence of particular strains in order to protect the vulnerable and minimise community spread. However, despite this, tens to hundreds of thousands of people around the world die from the flu annually.

There are no current effective treatments for COVID-19 and a lack of understanding of the underling immunopathology is a serious obstacle. Niacin has been proposed as potential supplement for COVID-19 patients based on earlier indications that chemically induced lung fibrosis was reduced in animal disease models. It was noted that Niacin inhibited Src kinase and P38MAP kinase activity and that alternate inhibitors of these enzymes were also efficacious for inhibiting MPO production in-vitro. One of these inhibitors, PPO also reduced ROS production under glucose challenge in a diabetic rat model as well as Palmitate induced ROS in a rat cell line.

Nicotinamide riboside (NR) (under the trade name Niagen®) or other NAD boosters are identified herein as therapeutics to use as part of a treatment to modulate the cytokine storm in COVID-19. The use of Niacin for suppression of NETosis is also set forth herein.

Genentech's Pulmozyme®, a nebulised DNASE-1, is used to degrade NETs formed in the lungs of cystic fibrosis patients and allows improved delivery of co-administered therapeutics. While the approach is effective in reducing mucus viscosity and preventing thrombosis. DNAse-1 can be deactivated through complexation with g-actin. Actin-resistant DNase (PRX-110/alidornase alfa) has given encouraging results in phase I and II clinical trials (ClinicalTrials.gov identifiers: NCT02605590, NCT02722122). Additionally, DNase 1—like 3n protein and other engineered DNases are being developed to dissolve NETs. However, NET degradation products retain their pro-inflammatory potential and can extend tissue damage and can release virus. Neutralizing the histone components of NETs with a protease or antibody-based approaches could help ameliorate these effects.

The present invention relates to a medical use of a pharmaceutical composition that includes one or more therapeutics to treat a respiratory disease caused by a viral infection. This includes an Acute Respiratory Distress Syndrome (ARDS). In another aspect of the present invention, the one or more therapeutics inhibits activation of a viral spike protein which results in the reduction or prevention of infectivity by the virus. In another aspect the viral infection is SARS-Coronavirus 2 (SARS CoV2). In a further aspect of the invention, the formulation of therapeutics reduces tissue damage and respiratory impairment induced by NETosis in response to infection.

Therefore, it would be a valuable to develop a single therapy or a combination therapy to treat both NETopathic events and simultaneously supress infectivity of viral pathogens, including SARS, MERS and COVID-19.

### SUMMARY OF THE INVENTION

The present invention provides a novel approach to treating and/or preventing severe, pathogenic immune response to viral infections by administering a combination of an inhibitor of Src family kinase-androgen receptor interaction and Niacin (i.e., vitamin B3). In one embodiment, the compositions and methods are used to treat a respiratory infection caused by a virus. In another embodiment, an infection is caused by bacteria of fungus.

In one embodiment, the viral infection is SARS-Coronavirus 2 (SARS CoV2). However, the compositions and methods can treat any disease that is mediated by inflammatory response to infection or sterile inflammation (i.e., sepsis and host directed NETopathy). Such diseases include COPD, Fibrosis, small vessel vasculitis, preeclampsia, endometriosis, psoriasis, gout, inflammatory bowel disease, ulcerative colitis, Chron's disease, anti-phospholipid syndrome, multiple sclerosis, sickle cell disease, lupus, arthritis, graft vs. host disease, organ transplant rejection, atherosclerosis, reperfusion injury, transfusion related lung injury (TRALI), Type 1 diabetes, Type 2 diabetes, obesity, Alzheimer's disease and gout as well as trauma such as blunt force injury and burns.

According to a first aspect, there is provided a therapeutic that inhibits or prevents an interaction between a Src family kinase and an androgen receptor (hereinafter, referred to as an “SA inhibitor”) for use in preventing or treating a disease in which a viral infection is mediated by androgen receptor activity in a subject. In a certain embodiment the SA inhibitor is a peptide whose sequence is derived from the SH3 binding domain of the human androgen receptor. In another embodiment the sequence is derived from the SH3 binding domain of an animal androgen receptor (AR). Such inhibitors are known in the art (US20100189776A1 and U.S. Ser. No. 10/023,612B2, whose entire disclosure are hereby incorporated by reference). In a particular embodiment the SA inhibitor comprises an isolated peptide containing the sequence Bj[(Pro)n-Xr-His-Pro-His-Ala-Arg-Ile-Lys]m-Rp (SEQ ID NO: 1) or a derivative or fragment thereof, wherein B is a first chemical moiety, j is 0 or 1, n is an integer from 1-10, X is any amino acid, r is an integer from 0 to 2, m is an integer from 1 to 3, R is a second chemical moiety, p is 0 or 1. In an embodiment, the SA inhibitor comprises an isolated peptide containing the sequence Ac-Pro-Pro-Pro-His-Pro-His-Ala-Arg-Ile-Lys-NH₂ (SEQ ID NO: 2). It will be clear to those in the art the sequence may be incorporated into a longer sequence as a derivative or fragment thereof. In an aspect, the SA inhibitor is capable of partially or completely blocking viral infectivity. In another aspect, the SA inhibitor is capable of blocking NETosis.

In certain embodiments the SA inhibitor is administered with a one, or more additional therapeutics. Amongst these therapeutics, the invention disclosed here include those that inhibit NETosis. In a further embodiment, the inhibitor of NETosis is a Niacin or a Niacin derivative. In another embodiment, the Niacin or Niacin derivate is a nicotinamide adenine dinucloeotide (NAD) or a NAD precursor. Therefore, in a further embodiment, the inhibitor of NETosis may include, but is not limited to, nicotinic acid (pyridine-3-carboxylic acid), nicotinamide (Niacinamide or pyridine-3-carboxamide) collectively known by the generic name Niacin (Vitamin B3) or nicotinamide riboside (1-(beta-D-Ribofuranosyl) nicotinamide). Additional embodiments, the Niacin derivative is formulated for extended duration of release, for example NIASPAN® (U.S. Pat. No. 6,818,229B, the entire disclosure of which relating to nicotinic acid formulations are hereby included by reference) and its generic equivalents.

In another embodiment the SA inhibitor is administered with a DNase enzyme, including a recombinant DNase or derivative thereof. In one embodiment, the recombinant DNase enzyme is Dornase Alpha. Several such enzymes are known in the art including, but not limited to, Pulmozyme® as well as its generic equivalents (see, e.g., U.S. Pat. No. 6,440,412B1, the entire disclosure of which is incorporated by reference). In another embodiment, the recombinant DNase is an Actin-resistant DNase (see e.g., U.S. Pat. No. 6,348,343B2, the entire disclosure of which is incorporated by reference). An example of an Actin-resistant DNase is. PRX-110/alidornase alfa.

In an embodiment, the SA inhibitor is administered to suppress viral infectivity with one, or more therapeutics that inhibit NETosis and a recombinant DNase enzyme.

In a further embodiment, there is provided a therapeutic that inhibits or prevents an interaction between a Src family kinase and an poly-proline rich motif from a pathogen.

In a further embodiment, there is provided a therapeutic that inhibits or prevents an interaction between a Src family kinase and a poly-proline rich motif from a virus (hereinafter, referred to as an “SA inhibitor”) for use in preventing or treating a disease in which a viral infection is mediated by binding of a viral protein containing the poly-proline rich motif to Src Homology 3 Domain of a host protein. In a certain embodiment the SA inhibitor is a peptide whose sequence is derived from the SH3 binding domain of the human androgen receptor. In another embodiment the sequence is derived from the SH3 binding domain of an animal androgen receptor (AR). Such inhibitors are known in the art (US20100189776A1 and U.S. Ser. No. 10/023,612B2, whose entire disclosure are hereby incorporated by reference). In a particular embodiment the SV inhibitor comprises an isolated peptide containing the sequence Bj[(Pro)n-Xr-His-Pro-His-Ala-Arg-Ile-Lys]m-Rp (SEQ ID NO: 1) or a derivative or fragment thereof, wherein B is a first chemical moiety, j is 0 or 1, n is an integer from 1-10, X is any amino acid, r is an integer from 0 to 2, m is an integer from 1 to 3, R is a second chemical moiety, p is 0 or 1. In an embodiment, the SV inhibitor comprises an isolated peptide containing the sequence Ac-Pro-Pro-Pro-His-Pro-His-Ala-Arg-Ile-Lys-NH₂ (SEQ ID NO: 2). It will be clear to those in the art the sequence may be incorporated into a longer sequence as a derivative or fragment thereof. In an aspect, the SV inhibitor is capable of partially or completely blocking viral replication.

In another embodiment, the SA inhibitor blocks other polyproline rich motifs (with similarity to the AR) from binding to SH3 domains.

In one embodiment the invention provides the use of a therapeutic that inhibits or prevents an interaction between a Src family kinase and an AR in the manufacture of a medicament for preventing or treating a disease in which a viral infection is mediated by androgen activity in a subject.

Similarly, the invention provides a method of preventing or treating a disease in which a viral infection is mediated by androgen activity in a subject

In another embodiment, the invention provides a method of preventing or treating a disease in which viral replication is mediated by recruitment of a host kinase following binding of a viral protein to an Src Homology 3 domain. It will be clear to those in the art that the Kinase can be any protein containing an SH3 domain. It will also be clear that inhibition of replication may be due to disruption of protein replication, packaging or assembly and release of live viruses.

In an embodiment, a pharmaceutical composition comprising an SA inhibitor is administered for preventing viral infection and associated NETopathic events. In an embodiment, a pharmaceutical composition comprising an SA inhibitor and a Niaspan® is administered for preventing viral infection and associated NETopathic events.

In a further embodiment, the SA inhibitor and NETosis inhibitor are co-administered with a recombinant DNase in a pharmaceutical composition. In an embodiment, a pharmaceutical composition comprising an SA inhibitor, a Niaspan® and Pulmozyme® is provided for preventing a viral infection and associated NETopathic events. In another embodiment, the viral infection is a respiratory viral infection.

### BRIEF DESCRIPTION OF THE DRAWINGS

FIG. 1A is a graph showing the results of dihydrotestosterone stimulation of LNCaP cells on the relative expression of ACE2 and TMPRSS2.

FIG. 1B is a graph showing the results of dihydrotestosterone stimulation of A459 cells on the relative expression of ACE2 and TMPRSS2

FIG. 2 is a blot showing the results of SA Inhibitor on TMPRSS2 protein expression in A549 cells in full media.

FIG. 3A is a blot and graph showing the results of SA Inhibitor on TMPRSS2 levels in LNCaP cells with dihydrotestosterone stimulation.

FIG. 3B is a blot and graph showing the results of SA Inhibitor on TMPRSS2 levels in A459 cells with dihydrotestosterone stimulation.

FIG. 4A is a graph showing SA inhibition of LPS triggered NETosis based on reduction of H3.1 nucleosomes.

FIG. 4B is a graph showing SA inhibition of LPS triggered NETosis based on reduction of H3.1 nucleosomes.

FIG. 4C is a graph showing SA inhibition of LPS triggered NETosis based on reduction of H3.1 nucleosomes.

FIG. 4D is a graph showing SA inhibition of LPS triggered NETosis based on reduction of H3.1 nucleosomes

### DEFINITIONS

Reference in this specification to “one embodiment/aspect” or “an embodiment/aspect” means that a particular feature, structure, or characteristic described in connection with the embodiment/aspect is included in at least one embodiment/aspect of the disclosure. The use of the phrase “in one embodiment/aspect” or “in another embodiment/aspect” in various places in the specification are not necessarily all referring to the same embodiment/aspect, nor are separate or alternative embodiments/aspects mutually exclusive of other embodiments/aspects. Moreover, various features are described which may be exhibited by some embodiments/aspects and not by others. Similarly, various requirements are described which may be requirements for some embodiments/aspects but not other embodiments/aspects. Embodiment and aspect can be in certain instances be used interchangeably.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. It will be appreciated that the same thing can be said in more than one way.

Consequently, alternative language and synonyms may be used for any one or more of the terms discussed herein. Nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

As applicable, the terms “about” or “generally”, as used herein in the specification and appended claims, and unless otherwise indicated, means a margin of +/−20%. Also, as applicable, the term “substantially” as used herein in the specification and appended claims, unless otherwise indicated, means a margin of +/−10%. It is to be appreciated that not all uses of the above terms are quantifiable such that the referenced ranges can be applied.

The term “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An active agent can be a primary active agent, or in other words, the component(s) of a composition to which the whole or part of the effect of the composition is attributed. An active agent can be a secondary agent, or in other words, the component(s) of a composition to which an additional part and/or other effect of the composition is attributed.

The term “coronavirus” refers to a group of related RNA viruses that cause diseases in mammals and birds. In humans, these viruses cause respiratory tract infections that can range from mild to lethal. Mild illnesses include some cases of the common cold (which is caused also by certain other viruses, predominantly rhinoviruses), while more lethal varieties can cause SARS, MERS, and COVID-19. There are presently no vaccines or antiviral drugs to prevent or treat human coronavirus infections.

The term “SARS” or “severe acute respiratory syndrome” refers to a viral respiratory disease of zoonotic origin that surfaced in the early 2000s caused by severe acute respiratory syndrome coronavirus (SARS-CoV or SARS-CoV-1), the first-identified strain of the SARS coronavirus species severe acute respiratory syndrome-related coronavirus (SARSr-CoV). The syndrome caused the 2002-2004 SARS outbreak. In 2019, its successor, the related virus strain Severe Acute Respiratory Syndrome Coronavirus 2 (SARS-CoV-2), was discovered.

The term “Covid-19” or “Coronavirus disease 2019” refers to a severe acute respiratory syndrome (SARS) caused by a virus known as SARS-Coronavirus 2 (SARS-CoV2).

The term “pharmaceutically acceptable carrier” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutically composition” as used herein refers to a composition comprising at least one compound as disclosed herein formulated together with one or more pharmaceutically acceptable carriers.

The term “viral load,” “viral burden” or “viral titer” refers to a numerical expression of the quantity of virus in a given volume of body fluid, usually blood plasma. It is often expressed as viral particles, or infectious particles per mL depending on the type of assay. A higher viral burden, titer, or viral load often correlates with the severity of an active viral infection.

As used herein, the term “recombinant” refers to polypeptides or polynucleotides that do not exist naturally and which may be created by combining polynucleotides or polypeptides in arrangements that would not normally occur together. The term can refer to a polypeptide produced through a biological host, selected from a mammalian expression system, an insect cell expression system, a yeast expression system, and a bacterial expression system.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only those polyclonal antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For example, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

The term “variant” as used herein includes modifications or chemical equivalents of the amino acid and nucleotide sequences disclosed herein that perform substantially the same function as the proteins or nucleic acid molecules disclosed herein in substantially the same way. For example, variants of proteins disclosed herein include, without limitation, conservative amino acid substitutions. Variants of proteins disclosed herein also include additions and deletions to the proteins disclosed herein. In addition, variant peptides and variant nucleotide sequences include analogs and chemical derivatives thereof.

The present therapeutic peptide can have amino acid additions, deletions, or substitutions. A modified amino acid sequence is a sequence that is different from the native amino acid sequence due to a deletion, an insertion, a non-conservative or conservative substitution or combinations thereof of one or more amino acid residues. In one embodiment, the modification is a point mutation. In one aspect, the modified therapeutic peptide does not have a naturally occurring sequence.

The amino acid substitutions may be conservative or non-conservative. A “conservative amino acid substitution”, as used herein, is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The most commonly occurring exchanges are Ala/Ser, Val/Ile, Asp/Glu, Thr/Ser, Ala/Gly, Ala/Thr, Ser/Asn, Ala/Val, Ser/Gly, Ala/Pro, Lys/Arg, Asp/Asn, Leu/Ile, Leu/Val, Ala/Glu and Asp/Gly, in both directions. Amino acid exchanges in proteins and peptides, which do not generally alter the activity of the proteins or peptides, are known in the art (H. Neurath, R. L. Hill, The Proteins, Academic Press, New York, 1979).

The term “derivative of a peptide” refers to a peptide having one or more residues chemically derivatized by reaction of a functional side group. Such derivatized molecules include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as derivatives are those peptides which contain one or more naturally occurring amino acid derivatives of the twenty standard amino acids. For examples: 4-hydroxyproline may be substituted for proline; 5-hydroxylysine may be substituted for lysine; 3-methylhistidine may be substituted for histidine; homoserine may be substituted for serine; and ornithine may be substituted for lysine.

In one embodiment, a modified therapeutic peptide disclosed herein can have 1-13 amino acid additions, deletions, or substitutions. In one aspect, the therapeutic peptide has at least 1, at least 2, at least 3, at least 4, at least 5, at least 6, at least 7, at least 8, at least 9, at least 10, at least 11, at least 12 or at least 13 amino acid additions, substitutions, or deletions. Substitutions can be conservative or non-conservative. In another aspect, the therapeutic peptide can have at most 13, at most 12, at most 11, at most 10, at most 9, at most 8, at most 7, at most 6, at most 5, at most 4, at most 3, at most 2, or at most 1 amino acid additions, substitutions, or deletions. In yet another aspect, the therapeutic peptide can have 1-13, 1-12, 1-10, 1-9, 1-8, 1-7, 1-6, 1-5, 1-4, 1-3, 1-2, 2-4, 2-13, 2-12, 2-10, 2-9, 2-8, 2-7, 2-6, 2-5, 2-4, 2-3, 3-4, 3-13, 3-12, 3-10, 3-9, 3-8, 3-7, 3-6, 3-5, 3-4, 4-12, 4-10, 4-9, 4-8, 4-7, 4-6, 4-5, 5-12, 5-10, 5-9, 5-8, 5-7, 5-6, 5-5, 6-12, 6-10, 6-9, 6-8, 6-7, 7-13, 7-12, 7-10, 7-9, 7-8, 8-13, 8-12, 8-10, 8-9, 9-13, 9-12, 9-10, 10-12, 11-13, 11-12 or 12-13 amino acid additions, substitutions or deletions.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to the full length of the reference sequence, usually about 25 to 100, or 50 to about 150, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (N) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

“Nucleic acid” refers to deoxyribonucleotides or ribonucleotides and polymers thereof in either single- or double-stranded form and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

A particular nucleic acid sequence also implicitly encompasses “splice variants.” Similarly, a particular protein encoded by a nucleic acid implicitly encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary but include alternate splicing of exons. Alternate polypeptides derived from the same nucleic acid by read-through transcription are also encompassed by this definition. Any products of a splicing reaction, including recombinant forms of the splice products, are included in this definition. An example of potassium channel splice variants is discussed in Leicher et al., J. Biol. Chem. 273(52):35095-35101 (1998).

As used herein, the term “prevention” means all of the actions by which the occurrence of the disease is restrained or retarded.

The term “treating” or “treatment” refers to one or more of (1) inhibiting the disease; e.g., inhibiting a disease, condition or disorder in an individual who is experiencing or displaying the pathology or symptomatology of the disease, condition or disorder (i.e., arresting further development of the pathology and/or symptomatology); and (2) ameliorating the disease; e.g., ameliorating a disease, condition or disorder in an individual who is experiencing or displaying the pathology or symptomatology of the disease, condition or disorder (i.e., reversing the pathology and/or symptomatology) such as decreasing the severity of disease.

The term “administration” refers to the introduction of an amount of a predetermined substance into a patient by a certain suitable method. The composition disclosed herein may be administered via any of the common routes, as long as it is able to reach a desired tissue, for example, but is not limited to, inhaling, intraperitoneal, intravenous, intramuscular, subcutaneous, intradermal, oral, topical, intranasal, intrapulmonary, or intrarectal administration. However, since peptides are digested upon oral administration, active ingredients of a composition for oral administration should be coated or formulated for protection against degradation in the stomach.

The term “individual,” “subject,” or “patient” refers to those who a susceptible to infection or who are suspected of having or diagnosed with an infectious disease. However, any patient to be treated with the pharmaceutical composition disclosed herein is included without limitation. The pharmaceutical composition including the peptide disclosed herein is administered to a patient to prevent and/or treat viral infection.

Construction of suitable vectors containing the desired sequences and control sequences employs standard ligation and restriction techniques, which are well understood in the art (see Maniatis et al., in Molecular Cloning: A Laboratory Manual, Cold Spring Harbor Laboratory, New York (1982)). Isolated plasmids, DNA sequences, or synthesized oligonucleotides are cleaved, tailored, and re-ligated in the form desired.

Other technical terms used herein have their ordinary meaning in the art that they are used, as exemplified by a variety of technical dictionaries. The particular values and configurations discussed in these non-limiting examples can be varied and are cited merely to illustrate at least one embodiment and are not intended to limit the scope thereof.

Preferably, the polypeptide domains in the therapeutic peptide are derived from the same host in which they are to be administered in order to reduce inflammatory responses against the administered therapeutic agents.

All numerical designations, e.g., pH, temperature, time, concentration, and molecular weight, including ranges, are to be understood as approximations in accordance with common practice in the art. When used herein, the term “about” may connote variation (+) or (−) 1%, 5% or 10% of the stated amount, as appropriate given the context. It is to be understood, although not always explicitly stated, that the reagents described herein are merely exemplary and that equivalents of such are known in the art.

Certain embodiments of the invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

A “pharmaceutical composition” can include the combination of an active agent, such as a therapeutic peptide, with a carrier, inert or active, in a sterile composition suitable for diagnostic or therapeutic use in vitro, in vivo or ex vivo.

### DETAILED DESCRIPTION

Embodiments of the invention include compositions and methods for treating or preventing severe, pathogenic immune response to viral infections. Specifically, a combination of an inhibitor of Src family kinase-androgen receptor interaction and Niacin are administered to a subject.

The invention is based on the recognition that treating NETopathic events associated with infection and simultaneously suppressing infectivity of viral pathogens can significantly reduce disease severity. The inventive combination has been found to be particularly useful in preventing the development of Acute Respiratory Distress Syndrome (ARDS) associated with Corona virus infection for a sufficient time to allow the host adaptive immune response to overcome the infection. For example, the combination is shown herein to be particularly suitable for treating COVID-19 resulting from infection with SARS-CoV-2. The invention not only provides methods of using the inventive combination of therapeutics but also includes pharmaceutical compositions and kits including the inventive combination.

In one embodiment, the compositions and methods are used to treat a respiratory infection caused by a virus. However, the compositions and methods described herein can be used to treat any disease mediated by inflammatory response to infection (which can be viral, bacterial or fungal) i.e. sepsis (which is described in the description) and host directed NETopathy which can result from sterile inflammation (including but not limited to multiple sclerosis, alzheimers, rheumatoid arthritis, vasculitis, preclampsia, graft vs host disease, transplant rejection as well as trauma—blunt force, burns).

Accordingly, in one aspect, a subject is treated by administering a combination of an SA inhibitor and Niacin.

The SA inhibitor can be a peptide whose sequence is derived from the SH3 binding domain of the human androgen receptor. In another embodiment the sequence is derived from the SH3 binding domain of an animal androgen receptor (AR). Such inhibitors are known in the art (see, e.g., US20100189776A1 and U.S. Ser. No. 10/023,612B2). In one embodiment the SA inhibitor comprises an isolated peptide with the following the sequence:

(SEQ ID NO: 1)
Bⱼ[(Pro)ₙ-Xr-His-Pro-His-Ala-Arg-Ile-Lys]ₘ-Rp

wherein B is a first chemical moiety, j is 0 or 1, n is an integer from 1-10, X is any amino acid, r is an integer from 0 to 2, m is an integer from 1 to 3, R is a second chemical moiety, p is 0 or 1.

In another embodiment, the SA inhibitor comprises an isolated peptide with the following sequence:

(SEQ ID NO: 2)
Ac-Pro-Pro-Pro-His-Pro-His-Ala-Arg-Ile-Lys-NH₂

It will be clear to those in the art either sequence can be incorporated into a longer sequence as well as a derivative or fragment thereof, including conservative variants. In one aspect, the SA inhibitor is capable of partially or completely blocking viral infectivity. In another aspect, the SA inhibitor is capable of blocking NETosis.

The SA inhibitor can be administered with a one, or more additional therapeutics. For example, the SA inhibitor can be co-administered with a therapeutic that inhibits or prevents NETosis. The inhibitor of NETosis can be, for example, Niacin or a Niacin derivative. In an embodiment, the Niacin or Niacin derivate is a nicotinamide adenine dinucloeotide (NAD) or a NAD precursor. Thus, the inhibitor of NETosis can include one or more of nicotinic acid (pyridine-3-carboxylic add), nicotinamide (Niacinamide or pyridine-3-carboxamide) collectively known by the generic name Niacin (Vitamin B3) or nicotinamide riboside (1-(beta-D-Ribofuranosyl) nicotinamide). The Niacin derivative can be formulated for extended duration of release, for example NIASPAN® (see, e.g., U.S. Pat. No. 6,818,229B) and its generic equivalents.

In another embodiment the SA inhibitor is administered with a DNase enzyme, including a recombinant DNase or derivative thereof. The recombinant DNase enzyme can be, for example, Dornase Alpha. Several such enzymes are known in the art including, Pulmozyme® and its generic equivalents (see, e.g., U.S. Pat. No. 6,440,412B1). In another embodiment, the recombinant DNase is an Actin-resistant DNase (see e.g., U.S. Pat. No. 6,348,343B2). An example of an Actin-resistant DNase is. PRX-110/alidornase alfa.

In one embodiment, the compositions have a therapeutic effect by their action in one of three modes. In a first mode, the SA inhibitor causes down regulation of transmembrane protease, serine 2 (TMPRSS2). The SA inhibitor host protease cleaves viral S glycoprotein to activate the virus synergistically with binding of the spike protein to ACE-2 receptors for cell entry. This process is similar to viral activation and cell entry of other coronaviruses, including SARS-CoV, as well as influenza virus such as influenza H1N1. TMPRSS2 activity is currently considered the sole protease crucial for cell entry and viral pathogenesis. This is described further in the below examples (i.e., Examples 1, 2 and 3).

The second mode involves down regulation of viral reproduction by the SA inhibitor. Certain viruses have been shown to “hijack” cellular machinery using proteins with proline rich motifs that bind to src Homology 3 Domains in a similar way to the Androgen receptor. Accordingly, in one embodiment, the invention provides a method of preventing or treating a disease in which viral replication is mediated by recruitment of a host kinase following binding of a viral protein to an Src Homology 3 domain. It will be clear to those in the art that the kinase can be any protein containing an SH3 domain. Inhibition of replication may be due to disruption of protein replication, packaging or assembly and release of live viruses. The SA inhibitor can inhibit or prevent this process which decreases viral reproduction.

The third mode involves down regulation of NETosis by the SA inhibitor. This is described further in the below examples (i.e., Examples 4 and 5).

In an embodiment, the combination of one or more therapeutics is useful in preventing the development of an Acute Respiratory Distress Syndrome (ARDS). The ARDS can be one that is associated with a Corona virus infection. In an embodiment, the pharmaceutical composition is provided for a sufficient amount of time to allow the individual to overcome the infection. In a further embodiment, the pharmaceutical composition treats an individual suffering from ARDS until the innate immune system is able to resolve the infection. In a further embodiment the combination is shown herein to be suitable for treating ARDS that may persist despite clearance of the underlying viral infection. For example, the combination is shown herein to be particularly suitable for treating an ARDS, including, COVID-19 resulting from infection with SARS-CoV-2. The use of one or more therapeutics can lead to an additive or a synergistic effect. In an embodiment, the mechanism of action occurs by inhibiting host directed NETopathic events and viral pathogens infectivity, resulting in a reduction of a diseases severity.

In an embodiment, inhibition of Src family kinase-Androgen Receptor (AR) interaction reduces cytoplasmic AR phosphorylation by Src kinase and induction of transcriptional activity. This has been reported for prostate and breast cancer and endometrial cells. See, for example, US20100189776A1 and U.S. Ser. No. 10/023,612B2, the entire disclosure of which is herein incorporated by reference. In another embodiment, inhibiting Src kinase-AR interaction reduces expression of a transmembrane protease, serine 2 (TMPRSS2). In a further embodiment inhibiting a Src kinase-AR interaction reduces expression of an ACE2. In a further embodiment inhibiting a Src kinase-AR interaction reduces expression of a Glucose Regulated Protein 78 (GPR78). There is provided a therapeutic that inhibits or prevents an interaction between a Src family kinase and an androgen receptor (the SA inhibitor) for use in preventing or treating a disease in which a viral infection is mediated by androgen receptor activity in a subject. In a certain embodiment the SA inhibitor is a peptide whose sequence is derived from the SH3 binding domain of the human androgen receptor. In another embodiment, the sequence is derived from the SH3 binding domain of an animal androgen receptor (AR). Such inhibitors are known in the art US20100189776A1 and U.S. Ser. No. 10/023,612B2. In an embodiment, the peptide sequence is Bj[(Pro)n-Xr-His-Pro-His-Ala-Arg-Ile-Lys]m-Rp (SEQ ID NO: 1) or a derivative or fragment thereof, wherein B is a first chemical moiety, j is 0 or 1, n is an integer from 1-10, X is any amino acid, r is an integer from 0 to 2, m is an integer from 1 to 3, R is a second chemical moiety, p is 0 or 1. In a further embodiment, the SA inhibitor comprises an isolated peptide containing the sequence Ac-Pro-Pro-Pro-His-Pro-His-Ala-Arg-Ile-Lys-NH₂ (SEQ ID NO: 2).

Src Family kinases are believed to be involved in NETosis in ex-vivo neutrophils stimulated with β-glucan (a component of bacterial, yeast and fungi cell walls). In an embodiment, this pathway is involved in viral stimulation of neutrophils. Therefore, in a further embodiment the invention provides a therapeutic that modulates the activity of Src family kinases for the prevention of NETosis in viral infections and ARDS infections. Examples of selective Src inhibitors include, 4-amino-5-(4-chlorophenyl)-7-(t-butyl) pyrazolo [3,4-d]pyrimidine (PP2) (PP2). In another embodiment, the inhibitor of Src kinase family interaction with AR also inhibits Src kinase family activation of NETosis. In an additional embodiment, the Src family kinase inhibitor comprises an isolated peptide containing the sequence Bj[(Pro)n-Xr-His-Pro-His-Ala-Arg-Ile-Lys]m-Rp or a derivative or fragment thereof, wherein B is a first chemical moiety, j is 0 or 1, n is an integer from 1-10, X is any amino acid, r is an integer from 0 to 2, m is an integer from 1 to 3, R is a second chemical moiety, p is 0 or 1. In a further embodiment, the peptide sequence is Ac-Pro-Pro-Pro-His-Pro-His-Ala-Arg-Ile-Lys-NH₂ (SEQ ID NO: 2). In an embodiment, an inhibitor of a SRC family kinase-AR interaction can also inhibit Src kinase mediated NETosis. Other therapeutics that also inhibit this interaction can also be useful for inhibiting NETosis, which includes those described in US20100189776A1 and U.S. Ser. No. 10/023,612B2, which are incorporated by reference.

In certain embodiments the SA inhibitor is administered with one, or more additional therapeutics that inhibit NETosis. In an embodiment, the inhibitor of NETosis is nicotinamide adenine dinucloeotide (NAD). In an embodiment, a precursor and/or a derivative of NAD may be used in place of NAD. Nicotinic acid or its derivative nicotinamide (two chemical forms of Niacin or vitamin B3) are chemical precursors to nicotinamide adenine dinucleotide (NAD), which is believed to suppresses NADPH oxidase activity and oxidative stress due to Reactive Oxygen Species (ROS) generation. Therefore, in a further embodiment, the inhibitor of NETosis may include, nicotinic acid (pyridine-3-carboxylic add), nicotinamide (Niacinamide or pyridine-3-carboxamide) collectively known by the generic name Niacin (Vitamin B3) and/or nicotinamide riboside (1-(beta-D-Ribofuranosyl) nicotinamide). In an embodiment, the Niacin derivative is formulated for extended duration of release, for example NIASPAN® as found in U.S. Pat. No. 6,818,229B, which is incorporated herein in its entirety, and which relates to nicotinic acid formulations and their generic equivalents. In an embodiment, alternate inhibitors of NETosis may further be combined with an inhibitor of a Src family kinase-AR interaction. Alternate inhibitors include, PAD4 inhibitors, Neutrophil Elastase Inhibitors, myeloperoxidase inhibitors

A combination of therapeutics can be used to ameliorate Netopathic effects in patients suffering from a viral respiratory infection or ARDS. For these patients, reducing the level of existing NETs can be beneficial. Therefore, in another embodiment the SA inhibitor is administered with a recombinant DNAse enzyme. In an embodiment, the recombinant DNase enzyme is Dornase Alpha. Several such enzymes are known in the art including, but not limited to, Pulmozyme®, as well as its generic equivalents. In another embodiment, the recombinant DNase is an Actin-resistant DNase as disclosed in U.S. Pat. No. 6,348,343B2, which is incorporated herein in its entirety. In another embodiment, the DNase enzyme is PRX-110/alidornase alfa.

In an embodiment, the SA inhibitor is administered with a second therapeutic, or a combination of therapeutics, that inhibit NETosis. In a further embodiment, the SA inhibitor is administered with a second therapeutic, or a combination of therapeutics, that inhibit NETosis, including a recombinant DNAse enzyme.

In one embodiment, the invention provides the use of a therapeutic that inhibits or prevents an interaction between a Src family kinase and an AR in the manufacture of a pharmaceutical composition for preventing or treating a disease in which a viral infection is mediated by androgen receptor activity in an individual.

Similarly, the invention provides a method of preventing or treating a disease in which a viral infection is mediated by androgen receptor activity in a subject.

In an embodiment, a pharmaceutical composition comprises an SA inhibitor and Niaspan® for preventing viral infection and associated NETopathic events.

In a further embodiment, an SA inhibitor and NETosis inhibitor are co-administered with a recombinant DNAse. In a further embodiment, a pharmaceutical composition comprising an SA inhibitor, Niaspan® and Pulmozyme are administered to an individual for the prevention of a viral infection and associated NETopathic events.

The compositions and methods described herein can be used to treat ailments that lead to NETopathic events including autoimmune diseases. Such autoimmune diseases include, for example, Addison's disease, celiac disease, dermatomyositis, fibromyalgia, Graves' disease, Guillain-Barre syndrome, Hashimoto thyroiditis, Kawasaki disease, multiple sclerosis, myasthenia gravis, pernicious anemia, psoriasis, reactive arthritis, rheumatic fever, rheumatoid arthritis, scleroderma, Sjögren syndrome, Systemic lupus erythematosus, type 1 diabetes, ulcerative colitis and vitiligo. COPD, Fibrosis, small vessel vasculitis, preeclampsia, endometriosis, psoriasis, gout, inflammatory bowel disease, Chron's disease, anti-phospholipid syndrome, sickle cell disease, graft vs host disease, organ transplant rejection, atherosclerosis, reperfusion injury, transfusion related lung injury (TRALI), Type 2 diabetes, Obesity, Alzheimer's Disease and Gout

Certain embodiments of the invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

As a general proposition, a therapeutically effective amount or prophylactically effective amount of a therapeutic peptide will be administered in a range from about 1 ng/kg body weight to about 100 mg/kg body weight whether by one or more administrations. In a particular embodiment, each therapeutic peptide is administered in the range of from about 1 ng/kg body weight to about 10 mg/kg body weight, about 1 ng/kg body weight to about 1 mg/kg body weight, about 1 ng/kg body weight to about 100 g/kg body weight, about 1 ng/kg body weight to about 10 g/kg body weight, about 1 ng/kg body weight/day to about 1 g/kg body weight, about 1 ng/kg body weight to about 100 ng/kg body weight, about 1 ng/kg body weight to about 10 ng/kg body weight, about 10 ng/kg body weight to about 100 mg/kg body weight, about 10 ng/kg body weight to about 10 mg/kg body weight, about 10 ng/kg body weight to about 1 mg/kg body weight, about 10 ng/kg body weight/to about 100 g/kg body weight, about 10 ng/kg body weight to about 10 mg/kg body weight, about 10 ng/kg body weight to about 1 mg/kg body weight, 10 ng/kg body weight to about 100 ng/kg body weight/, about 100 ng/kg body weight to about 100 mg/kg body weight, about 100 ng/kg body weight to about 10 mg/kg body weight, about 100 ng/kg body weight to about 1 mg/kg body weight, about 100 ng/kg body weight to about 100 mg/kg body weight, about 100 ng/kg body weight to about 10 mg/kg body weight, about 100 ng/kg body weight to about 1 mg/kg body weight, about 1 mg/kg body weight to about 100 mg/kg body weight, about 1 mg/kg body weight to about 10 mg/kg body weight/day, about 1 mg/kg body weight to about 1 mg/kg body weight, about 1 mg/kg body weight to about 100 mg/kg body weight, about 1 mg/kg body weight to about 10 mg/kg body weight, about 10 mg/kg body weight to about 100 mg/kg body weight, about 10 mg/kg body weight to about 10 mg/kg body weight, about 10 mg/kg body weight to about 1 mg/kg body weight/day, about 10 mg/kg body weight to about 100 mg/kg body weight, about 100 mg/kg body weight/day to about 100 mg/kg body weight, about 100 mg/kg body weight/day to about 10 mg/kg body weight, about 100 mg/kg body weight/day to about 1 mg/kg body weight, about 1 mg/kg body weight to about 100 mg/kg body weight, about 1 mg/kg body weight to about 10 mg/kg body weight, about 10 mg/kg body weight to about 100 mg/kg body weight/day.

In other embodiments, a therapeutic peptide is administered in the range of about 10 ng to about 100 ng per individual administration, about 10 ng to about 1 g per individual administration, about 10 ng to about 10 g per individual administration, about 10 ng to about 100 mg per individual administration, about 10 ng to about 1 mg per individual administration, about 10 ng to about 10 mg per individual administration, about 10 ng to about 100 mg per individual administration, about 10 ng to about 1000 mg per injection, about 10 ng to about 10,000 mg per individual administration, about 100 ng to about 1 mg per individual administration, about 100 ng to about 10 mg per individual administration, about 100 ng to about 100 mg per individual administration, about 100 ng to about 1 mg per individual administration, about 100 ng to about 10 mg per individual administration, about 100 ng to about 100 mg per individual administration, about 100 ng to about 1000 mg per injection, about 100 ng to about 10,000 mg per individual administration, about 1 mg to about 10 mg per individual administration, about 1 mg to about 100 mg per individual administration, about 1 mg to about 1 mg per individual administration, about 1 mg to about 10 mg per individual administration, about 1 mg to about 100 mg per individual administration, about 1 mg to about 1000 mg per injection, about 1 mg to about 10,000 mg per individual administration, about 10 mg to about 100 mg per individual administration, about 10 mg to about 1 mg per individual administration, about 10 mg to about 10 mg per individual administration, about 10 mg to about 100 mg per individual administration, about 10 mg to about 1000 mg per injection, about 10 mg to about 10,000 mg per individual administration, about 100 mg to about 1 mg per individual administration, about 100 mg to about 10 mg per individual administration, about 100 mg to about 100 mg per individual administration, about 100 mg to about 1000 mg per injection, about 100 mg to about 10,000 mg per individual administration, about 1 mg to about 10 mg per individual administration, about 1 mg to about 100 mg per individual administration, about 1 mg to about 1000 mg per injection, about 1 mg to about 10,000 mg per individual administration, about 10 mg to about 100 mg per individual administration, about 10 mg to about 1000 mg per injection, about 10 mg to about 10,000 mg per individual administration, about 100 mg to about 1000 mg per injection, about 100 mg to about 10,000 mg per individual administration and about 1000 mg to about 10,000 mg per individual administration. The therapeutic peptide may be administered daily, every 2, 3, 4, 5, 6 or 7 days, or every 1, 2, 3 or 4 weeks.

In other particular embodiments, the amount of the therapeutic peptide can be administered at a dose of about 0.0006 mg, 0.001 mg, 0.003 mg, 0.006 mg, 0.01 mg, 0.03 mg, 0.06 mg, 0.1 mg, 0.3 mg, 0.6 mg, 1 mg, 3 mg, 6 mg, 10 mg, 30 mg, 60 mg, 100 mg, 300 mg, 600 mg, 1000 mg, 2000 mg, 5000 mg or 10,000 mg. As expected, the dosage will be dependent on the condition, size, age and condition of the patient.

In other aspects of this embodiment, a pharmaceutical composition compound disclosed herein reduces the incidence of viral infection by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In yet other aspects of this embodiment, a pharmaceutical composition disclosed herein reduces the incidence of viral infection from, e.g., about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

A pharmaceutical composition disclosed herein is in an amount sufficient to allow customary administration to an individual. In aspects of this embodiment, a pharmaceutical composition disclosed herein can be, e.g., at least 5 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, or at least 100 mg of each therapeutic. In other aspects of this embodiment, a pharmaceutical composition disclosed herein may be, e.g., at least 5 mg, at least 10 mg, at least 20 mg, at least 25 mg, at least 50 mg, at least 75 mg, at least 100 mg, at least 200 mg, at least 300 mg, at least 400 mg, at least 500 mg, at least 600 mg, at least 700 mg, at least 800 mg, at least 900 mg, at least 1,000 mg, at least 1,100 mg, at least 1,200 mg, at least 1,300 mg, at least 1,400 mg, or at least 1,500 mg of each therapeutic. In yet other aspects of this embodiment, a pharmaceutical composition disclosed herein may be in the range of, e.g., about 5 mg to about 100 mg, about 10 mg to about 100 mg, about 50 mg to about 150 mg, about 100 mg to about 250 mg, about 150 mg to about 350 mg, about 250 mg to about 500 mg, about 350 mg to about 600 mg, about 500 mg to about 750 mg, about 600 mg to about 900 mg, about 750 mg to about 1,000 mg, about 850 mg to about 1,200 mg, or about 1,000 mg to about 1,500 mg each therapeutic. In still other aspects of this embodiment, a pharmaceutical composition disclosed herein may be in the range of, e.g., about 10 mg to about 250 mg, about 10 mg to about 500 mg, about 10 mg to about 750 mg, about 10 mg to about 1,000 mg, about 10 mg to about 1,500 mg, about 50 mg to about 250 mg, about 50 mg to about 500 mg, about 50 mg to about 750 mg, about 50 mg to about 1,000 mg, about 50 mg to about 1,500 mg, about 100 mg to about 250 mg, about 100 mg to about 500 mg, about 100 mg to about 750 mg, about 100 mg to about 1,000 mg, about 100 mg to about 1,500 mg, about 200 mg to about 500 mg, about 200 mg to about 750 mg, about 200 mg to about 1,000 mg, about 200 mg to about 1,500 mg, about 5 mg to about 1,500 mg, about 5 mg to about 1,000 mg, or about 5 mg to about 250 mg each therapeutic.

A pharmaceutical composition disclosed herein can comprise a solvent, emulsion or other diluent in an amount sufficient to dissolve a pharmaceutical composition disclosed herein. In other aspects of this embodiment, a pharmaceutical composition disclosed herein may comprise a solvent, emulsion or a diluent in an amount of, e.g., less than about 90% (v/v), less than about 80% (v/v), less than about 70% (v/v), less than about 65% (v/v), less than about 60% (v/v), less than about 55% (v/v), less than about 50% (v/v), less than about 45% (v/v), less than about 40% (v/v), less than about 35% (v/v), less than about 30% (v/v), less than about 25% (v/v), less than about 20% (v/v), less than about 15% (v/v), less than about 10% (v/v), less than about 5% (v/v), or less than about 1% (v/v). In other aspects of this embodiment, a pharmaceutical composition disclosed herein may comprise a solvent, emulsion or other diluent in an amount in a range of, e.g., about 1% (v/v) to 90% (v/v), about 1% (v/v) to 70% (v/v), about 1% (v/v) to 60% (v/v), about 1% (v/v) to 50% (v/v), about 1% (v/v) to 40% (v/v), about 1% (v/v) to 30% (v/v), about 1% (v/v) to 20% (v/v), about 1% (v/v) to 10% (v/v), about 2% (v/v) to 50% (v/v), about 2% (v/v) to 40% (v/v), about 2% (v/v) to 30% (v/v), about 2% (v/v) to 20% (v/v), about 2% (v/v) to 10% (v/v), about 4% (v/v) to 50% (v/v), about 4% (v/v) to 40% (v/v), about 4% (v/v) to 30% (v/v), about 4% (v/v) to 20% (v/v), about 4% (v/v) to 10% (v/v), about 6% (v/v) to 50% (v/v), about 6% (v/v) to 40% (v/v), about 6% (v/v) to 30% (v/v), about 6% (v/v) to 20% (v/v), about 6% (v/v) to 10% (v/v), about 8% (v/v) to 50% (v/v), about 8% (v/v) to 40% (v/v), about 8% (v/v) to 30% (v/v), about 8% (v/v) to 20% (v/v), about 8% (v/v) to 15% (v/v), or about 8% (v/v) to 12% (v/v).

The final concentration of a pharmaceutical composition disclosed herein can be of any concentration desired. In an aspect of this embodiment, the final concentration of each therapeutic in a pharmaceutical composition may be a therapeutically effective amount. In other aspects of this embodiment, the final concentration of each therapeutic of a pharmaceutical composition may be, e.g., at least 0.00001 mg/mL, at least 0.0001 mg/mL, at least 0.001 mg/mL, at least 0.01 mg/mL, at least 0.1 mg/mL, at least 1 mg/mL, at least 10 mg/mL, at least 25 mg/mL, at least 50 mg/mL, at least 100 mg/mL, at least 200 mg/mL or at least 500 mg/mL. In other aspects of this embodiment, the final concentration of each therapeutic of a pharmaceutical composition may be in a range of, e.g., about 0.00001 mg/mL to about 3,000 mg/mL, about 0.0001 mg/mL to about 3,000 mg/mL, about 0.01 mg/mL to about 3,000 mg/mL, about 0.1 mg/mL to about 3,000 mg/mL, about 1 mg/mL to about 3,000 mg/mL, about 250 mg/mL to about 3,000 mg/mL, about 500 mg/mL to about 3,000 mg/mL, about 750 mg/mL to about 3,000 mg/mL, about 1,000 mg/mL to about 3,000 mg/mL, about 100 mg/mL to about 2,000 mg/mL, about 250 mg/mL to about 2,000 mg/mL, about 500 mg/mL to about 2,000 mg/mL, about 750 mg/mL to about 2,000 mg/mL, about 1,000 mg/mL to about 2,000 mg/mL, about 100 mg/mL to about 1,500 mg/mL, about 250 mg/mL to about 1,500 mg/mL, about 500 mg/mL to about 1,500 mg/mL, about 750 mg/mL to about 1,500 mg/mL, about 1,000 mg/mL to about 1,500 mg/mL, about 100 mg/mL to about 1,200 mg/mL, about 250 mg/mL to about 1,200 mg/mL, about 500 mg/mL to about 1,200 mg/mL, about 750 mg/mL to about 1,200 mg/mL, about 1,000 mg/mL to about 1,200 mg/mL, about 100 mg/mL to about 1,000 mg/mL, about 250 mg/mL to about 1,000 mg/mL, about 500 mg/mL to about 1,000 mg/mL, about 750 mg/mL to about 1,000 mg/mL, about 100 mg/mL to about 750 mg/mL, about 250 mg/mL to about 750 mg/mL, about 500 mg/mL to about 750 mg/mL, about 100 mg/mL to about 500 mg/mL, about 250 mg/mL to about 500 mg/mL, about 0.00001 mg/mL to about 0.0001 mg/mL, about 0.00001 mg/mL to about 0.001 mg/mL, about 0.00001 mg/mL to about 0.01 mg/mL, about 0.00001 mg/mL to about 0.1 mg/mL, about 0.00001 mg/mL to about 1 mg/mL, about 0.001 mg/mL to about 0.01 mg/mL, about 0.001 mg/mL to about 0.1 mg/mL, about 0.001 mg/mL to about 1 mg/mL, about 0.001 mg/mL to about 10 mg/mL, or about 0.001 mg/mL to about 100 mg/mL.

Aspects of the present specification disclose, in part, treating an individual who is susceptible to viral infection or suffering from viral infection. As used herein, the term “treating,” refers to reducing or eliminating the incidence of viral infection; or lowering or depleting the viral load. For example, the term “treating” can mean reducing a symptom of a condition characterized by a viral infection, including, but not limited to, decreasing viral load, by, e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 100%. Those of skill in the art will know the appropriate symptoms or indicators associated with a specific type of ailment and will know how to determine if an individual is a candidate for treatment as disclosed herein.

Aspects of the present also disclose treating an individual who is susceptible to a disease that can manifest in an inflammatory response through NETosis. As used herein, the term “treating,” refers to reducing or eliminating the signs/symptoms of a disease. For example, the term “treating” can mean reducing a symptom of a condition characterized by a disease, by, e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 100%. Those of skill in the art will know the appropriate symptoms or indicators associated with a specific type of ailment and will know how to determine if an individual is a candidate for treatment as disclosed herein.

In aspects of this embodiment, a therapeutically effective amount of a pharmaceutical composition disclosed herein reduces viral load, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a therapeutically effective amount of a pharmaceutical composition disclosed herein reduces viral load by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutically effective amount of a pharmaceutical composition disclosed herein reduces viral load by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In aspects of this embodiment, a therapeutically effective amount of a pharmaceutical composition disclosed herein reduces a sign/symptom of a disease by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a therapeutically effective amount of a pharmaceutical composition disclosed herein reduces a sign/symptom by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutically effective amount of a pharmaceutical composition disclosed herein reduces a sign/symptom by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In yet other aspects of this embodiment, a therapeutically effective amount of each therapeutic of a pharmaceutical composition disclosed herein generally is in the range of about 0.001 mg/kg to about 100 mg/kg and administered, for example, every 3, 5, 7, 10 or 14 days. In aspects of this embodiment, an effective amount of each therapeutic of a pharmaceutical composition disclosed herein may be, e.g., at least 0.001 mg/kg, at least 0.01 mg/kg, at least 0.1 mg/kg, at least 1.0 mg/kg, at least 5.0 mg/kg, at least 10 mg/kg, at least 15 mg/kg, at least 20 mg/kg, at least 25 mg/kg, at least 30 mg/kg, at least 35 mg/kg, at least 40 mg/kg, at least 45 mg/kg, or at least 50 mg/kg and administered, for example, every 3, 5, 7, 10 or 14 days. In other aspects of this embodiment, an effective amount of each therapeutic of a pharmaceutical composition disclosed herein may be in the range of, e.g., about 0.001 mg/kg to about 10 mg/kg, about 0.001 mg/kg/day to about 15 mg/kg, about 0.001 mg/kg to about 20 mg/kg, about 0.001 mg/kg to about 25 mg/kg, about 0.001 mg/kg to about 30 mg/kg, about 0.001 mg/kg to about 35 mg/kg, about 0.001 mg/kg to about 40 mg/kg, about 0.001 mg/kg to about 45 mg/kg, about 0.001 mg/kg to about 50 mg/kg, about 0.001 mg/kg to about 75 mg/kg, or about 0.001 mg/kg to about 100 mg/kg and administered, for example, every 3, 5, 7, 10 or 14 days. In yet other aspects of this embodiment, an effective amount of each therapeutic of a pharmaceutical composition disclosed herein may be in the range of, e.g., about 0.01 mg/kg to about 10 mg/kg, about 0.01 mg/kg to about 15 mg/kg, about 0.01 mg/kg to about 20 mg/kg, about 0.01 mg/kg to about 25 mg/kg, about 0.01 mg/kg to about 30 mg/kg, about 0.01 mg/kg to about 35 mg/kg, about 0.01 mg/kg to about 40 mg/kg, about 0.01 mg/kg to about 45 mg/kg, about 0.01 mg/kg to about 50 mg/kg, about 0.01 mg/kg to about 75 mg/kg, or about 0.01 mg/kg to about 100 mg/kg and administered, for example, every 3, 5, 7, 10 or 14 days. In still other aspects of this embodiment, an effective amount of each therapeutic of a pharmaceutical composition disclosed herein may be in the range of, e.g., about 0.1 mg/kg to about 10 mg/kg, about 0.1 mg/kg to about 15 mg/kg, about 0.1 mg/kg to about 20 mg/kg, about 0.1 mg/kg to about 25 mg/kg, about 0.1 mg/kg to about 30 mg/kg, about 0.1 mg/kg to about 35 mg/kg, about 0.1 mg/kg to about 40 mg/kg, about 0.1 mg/kg to about 45 mg/kg, about 0.1 mg/kg to about 50 mg/kg, about 0.1 mg/kg to about 75 mg/kg, or about 0.1 mg/kg to about 100 mg/kg and administered, for example, every 3, 5, 7, 10 or 14 days.

In one embodiment, a pharmaceutical composition disclosed herein is capable of reducing the incidence of viral infection by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment. In other aspects of this embodiment, viral load in an individual is decreased by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to a patient not receiving the same treatment.

In a further embodiment, the therapeutic peptide and its derivatives have half-lives of 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 1 week, 2 weeks, 3 weeks, 4 weeks, one month, two months, three months, four months or more.

In aspects of this embodiment, a therapeutically effective amount of a therapeutic disclosed herein reduces the incidence of viral infection by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a therapeutically effective amount of a therapeutic disclosed herein reduces the incidence of viral infection by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutically effective amount of a therapeutic disclosed herein reduces the incidence of viral infection by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In one embodiment, the dose of the composition may be administered daily, semi-weekly, weekly, bi-weekly, or monthly. The period of treatment may be for a week, two weeks, a month, two months, four months, six months, eight months, a year, or longer. The initial dose may be larger than a sustaining dose. In one embodiment, the dose ranges from a weekly dose of at least 0.01 mg/kg, at least 0.25 mg/kg, at least 0.3 mg/kg, at least 0.5 mg/kg, at least 0.75 mg/kg, at least 1 mg/kg, at least 2 mg/kg, at least 3 mg/kg, at least 4 mg/kg, at least 5 mg/kg, at least 6 mg/kg, at least 7 mg/kg, at least 8 mg/kg, at least 9 mg/kg, at least 10 mg/kg, at least 15 mg/kg, at least 20 mg/kg, at least 25 mg/kg, or at least 30 mg/kg In one embodiment, a weekly dose may be at most 1.5 mg/kg, at most 2 mg/kg, at most 2.5 mg/kg, at most 3 mg/kg, at most 4 mg/kg, at most 5 mg/kg, at most 6 mg/kg, at most 7 mg/kg, at most 8 mg/kg, at most 9 mg/kg, at most 10 mg/kg, at most 15 mg/kg, at most 20 mg/kg, at most 25 mg/kg, or at most 30 mg/kg. In a particular aspect, the weekly dose may range from 5 mg/kg to 20 mg/kg. In an alternative aspect, the weekly dose may range from 10 mg/kg to 15 mg/kg.

The present specification also provides a pharmaceutical composition for the administration to a subject. The pharmaceutical composition disclosed herein may further include a pharmaceutically acceptable carrier, excipient, or diluent. As used herein, the term “pharmaceutically acceptable” means that the composition is sufficient to achieve the therapeutic effects without deleterious side effects, and may be readily determined depending on the type of the diseases, the patient's age, body weight, health conditions, gender, and drug sensitivity, administration route, administration mode, administration frequency, duration of treatment, drugs used in combination or coincident with the composition disclosed herein, and other factors known in medicine.

Moreover, the pharmaceutical composition may be administered alone or in combination or coincident with other pharmaceutical formulations showing prophylactic or therapeutic efficacy.

Given the teachings and guidance provided herein, those skilled in the art will understand that a formulation described herein can be equally applicable to many types of biopharmaceuticals, including those exemplified, as well as others known in the art. Given the teachings and guidance provided herein, those skilled in the art also will understand that the selection of, for example, type(s) or and/or amount(s) of one or more excipients, surfactants and/or optional components can be made based on the chemical and functional compatibility with the biopharmaceutical to be formulated and/or the mode of administration as well as other chemical, functional, physiological and/or medical factors well known in the art. For example, non-reducing sugars exhibit favorable excipient properties when used with polypeptide biopharmaceuticals compared to reducing sugars. Accordingly, exemplary formulations are exemplified further herein with reference to polypeptide biopharmaceuticals. However, the range of applicability, chemical and physical properties, considerations and methodology applied to polypeptide biopharmaceutical can be similarly applicable to biopharmaceuticals other than polypeptide biopharmaceuticals.

In various embodiments, a formulation can include, without limitation, combinations of bioactive agents (such as viruses, proteins, antibodies, peptides and the like as described herein) in the formulation. For example, a formulation as described herein can include a single bioactive agent for treatment of one or more conditions, including without limitation, disease. A formulation as described herein also can include, in an embodiment, without limitation, two or more different bioactive agents for a single or multiple conditions. Use of multiple bioactive agents in a formulation can be directed to, for example, the same or different indications. Similarly, in another embodiment, multiple bioactive agents can be used in a formulation to treat, for example, both a pathological condition and one or more side effects caused by the primary treatment. In a further embodiment, multiple bioactive agents also can be included, without limitation, in a formulation as described herein to accomplish different medical purposes including, for example, simultaneous treatment and monitoring of the progression of the pathological condition. In an additional embodiment, multiple, concurrent therapies such as those exemplified herein as well as other combinations well known in the art are particularly useful for patient compliance because a single formulation can be sufficient for some or all suggested treatments and/or diagnosis. Those skilled in the art will know those bioactive agents that can be admixed for a wide range of combination therapies. Similarly, in various embodiments, a formulation can be used with a small molecule drug and combinations of one or more bioactive agents together with one or more small molecule pharmaceuticals. Therefore, in various embodiments a formulation is provided containing 1, 2, 3, 4, 5 or 6 or more different bioactive agents, as well as, for one or more bioactive agents combined with one or more small molecule pharmaceuticals.

The composition can therefore be administered as a single dose, or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages may be ascertained through use of appropriate dose-response data. In various embodiments, the bioactive agents in formulations described herein can, without limitation, be administered to patients throughout an extended time period, such as chronic administration for a chronic condition. The composition can be a solid, a semi-solid or an aerosol and a pharmaceutical compositions is formulated as a tablet, geltab, lozenge, orally dissolved strip, capsule, syrup, oral suspension, emulsion, granule, sprinkle or pellet.

In an embodiment, for oral, rectal, vaginal, parenteral, pulmonary, sublingual and/or intranasal delivery formulations, tablets can be made by compression or molding, optionally with one or more accessory ingredients or additives. In an embodiment, compressed tablets are prepared, for example, by compressing in a suitable tabletting machine, the active ingredients in a free-flowing form such as a powder or granules, optionally mixed with a binder (for example, without limitation, povidone, gelatin, hydroxypropylmethyl cellulose), lubricant, inert diluent, preservative, disintegrant (for example, without limitation, sodium starch glycolate, cross-linked povidone, cross-linked sodium carboxymethyl cellulose) and/or surface-active or dispersing agent.

Packaging and instruments for administration may be determined by a variety of considerations, such as, without limitation, the volume of material to be administered, the conditions for storage, whether skilled healthcare practitioners will administer or patient self-compliance, the dosage regime, the geopolitical environment (e.g., exposure to extreme conditions of temperature for developing nations), and other practical considerations.

Injection devices include pen injectors, auto injectors, safety syringes, injection pumps, infusion pumps, glass prefilled syringes, plastic prefilled syringes and needle free injectors syringes may be prefilled with liquid, or may be dual chambered, for example, for use with lyophilized material. An example of a syringe for such use is the Lyo-Ject™, a dual-chamber pre-filled lyosyringe available from Vetter GmbH, Ravensburg, Germany. Another example is the LyoTip which is a prefilled syringe designed to conveniently deliver lyophilized formulations available from LyoTip, Inc., Camarillo, Calif., U.S.A. Administration by injection may be, without limitation intravenous, intramuscular, intraperitoneal, or subcutaneous, as appropriate. Administrations by non-injection route may be, without limitation, nasal, oral, cocular, dermal, or pulmonary, as appropriate.

In certain embodiments, kits can comprise, without limitation, one or more single or multi-chambered syringes (e.g., liquid syringes and lyosyringes) for administering one or more formulations described herein. In various embodiments, the kit can comprise formulation components for parenteral, subcutaneous, intramuscular or IV administration, sealed in a vial under partial vacuum in a form ready for loading into a syringe and administration to a subject. In this regard, the composition can be disposed therein under partial vacuum. In all of these embodiments and others, the kits can contain one or more vials in accordance with any of the foregoing, wherein each vial contains a single unit dose for administration to a subject.

The kits can comprise lyophilates, disposed as herein, that upon reconstitution provide compositions in accordance therewith. In various embodiment the kits can contain a lyophilate and a sterile diluent for reconstituting the lyophilate. The kit can also contain instructions for the use of the pharmaceutical composition. If the pharmaceutical composition is in a dry form, for example, a lyophlate, the kit can also contain diluent to dissolve the pharmaceutical composition.

Also described herein, are methods for treating a subject in need of therapy, comprising administering to the subject an effective amount of a formulation as described herein. The therapeutically effective amount or dose of a formulation will depend on the disease or condition of the subject and actual clinical setting.

In an embodiment, a formulation as described herein can be administered by any suitable route, specifically by parental (including subcutaneous, intramuscular, intravenous and intradermal) administration. It will also be appreciated that the preferred route will vary with the condition and age of the recipient, and the disease being treated. Methods of determining the most effective means and dosage of administration are known to those of skill in the art and will vary, without limitation, with the composition used for therapy, the purpose of the therapy, and the subject being treated. Single or multiple administrations can be carried out, without limitation, the dose level and pattern being selected by the treating physician. Suitable dosage formulations and methods of administering the agents are known in the art.

The formulations as described herein can be used in the manufacture of medicaments and for the treatment of humans and other animals by administration in accordance with conventional procedures.

Also provided herein are combinatorial methods for developing suitable virus formulations using combinations of amino acids. These methods are effective for developing stable liquid or lyophilized formulations, and particularly pharmaceutical virus formulations.

Compositions in accordance with embodiments described herein have desirable properties, such as desirable solubility, viscosity, syringeability and stability. Lyophilates in accordance with embodiments described herein have desirable properties, as well, such as desirable recovery, stability and reconstitution.

In an embodiment, the pH of the pharmaceutical formulation is at least about 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, or 9.

In an embodiment, the pH of the pharmaceutical formulation is from about 3 to about 9, about 4 to about 19, about 5 to about 9, about 6 to about 8, about 6 to about 7, about 6 to about 9, about 5 to about 6, about 5 to about 7, about 5 to about 8, about 4 to about 9, about 4 to about 8, about 4 to about 7, about 4 to about 6, about 4 to about 5, about 3 to about 8, about 3 to about 7, about 3 to about 6, about 3 to about 5, about 3 to about 4, about 7 to about 8, about 7 to about 9, about 7 to about 10.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

In an embodiment, the additive or synergistic interaction from a combination of a Niacin or Niacin derivative and an inhibitor of a Src family kinase-androgen receptor in the treatment of ARDS associated with a Corona virus infection has been demonstrated herein. In other embodiments the inventive combination has been found to be particularly useful in preventing the development of ARDS for a sufficient time to allow the host adaptive immune response to overcome the infection. For example, the combination is shown herein to be particularly suitable for treating COVID-19 resulting from infection with SARS-CoV-2.

The invention not only provides methods of using the inventive combination of therapeutics but also includes pharmaceutical compositions and kits including the inventive combination.

A pharmaceutical composition disclosed herein may optionally include a pharmaceutically-acceptable carrier that facilitates processing of an active ingredient into pharmaceutically-acceptable compositions. As used herein, the term “pharmacologically-acceptable carrier” is synonymous with “pharmacological carrier” and means any carrier that has substantially no long term or permanent detrimental effect when administered and encompasses terms such as “pharmacologically acceptable vehicle, stabilizer, diluent, additive, auxiliary or excipient.” Such a carrier generally is mixed with an active compound or permitted to dilute or enclose the active compound and can be a solid, semi-solid, or liquid agent. It is understood that the active ingredients can be soluble or can be delivered as a suspension in the desired carrier or diluent. Any of a variety of pharmaceutically acceptable carriers can be used including, without limitation, aqueous media such as, e.g., water, saline, glycine, hyaluronic acid and the like; solid carriers such as, e.g., mannitol, lactose, starch, magnesium stearate, sodium saccharin, talcum, cellulose, glucose, sucrose, magnesium carbonate, and the like; solvents; dispersion media; coatings; antibacterial and antifungal agents; isotonic and absorption delaying agents; or any other inactive ingredient. Selection of a pharmacologically acceptable carrier can depend on the mode of administration. Except insofar as any pharmacologically acceptable carrier is incompatible with the active ingredient, its use in pharmaceutically acceptable compositions is contemplated. Non-limiting examples of specific uses of such pharmaceutical carriers can be found in Pharmaceutical Dosage Forms and Drug Delivery Systems (Howard C. Ansel et al., eds., Lippincott Williams & Wilkins Publishers, 7th ed. 1999); REMINGTON: THE SCIENCE AND PRACTICE OF PHARMACY (Alfonso R. Gennaro ed., Lippincott, Williams & Wilkins, 20th ed. 2000); Goodman & Gilman's The Pharmacological Basis of Therapeutics (Joel G. Hardman et al., eds., McGraw-Hill Professional, 10th ed. 2001); and Handbook of Pharmaceutical Excipients (Raymond C. Rowe et al., APhA Publications, 4th edition 2003). These protocols are routine procedures and any modifications are well within the scope of one skilled in the art and from the teaching herein.

A pharmaceutical composition disclosed herein can optionally include, without limitation, other pharmaceutically acceptable components (or pharmaceutical components), including, without limitation, buffers, preservatives, tonicity adjusters, salts, antioxidants, osmolality adjusting agents, physiological substances, pharmacological substances, bulking agents, emulsifying agents, wetting agents, flavoring agents, coloring agents, and the like. Various buffers and means for adjusting pH can be used to prepare a pharmaceutical composition disclosed herein, provided that the resulting preparation is pharmaceutically acceptable. Such buffers include, without limitation, acetate buffers, citrate buffers, phosphate buffers, neutral buffered saline, phosphate buffered saline and borate buffers. It is understood that acids or bases can be used to adjust the pH of a composition as needed. Pharmaceutically acceptable antioxidants include, without limitation, sodium metabisulfite, sodium thiosulfate, acetylcysteine, butylated hydroxyanisole and butylated hydroxytoluene. Useful preservatives include, without limitation, benzalkonium chloride, chlorobutanol, thimerosal, phenylmercuric acetate, phenylmercuric nitrate, a stabilized oxy chloro composition and chelants, such as, e.g., DTPA or DTPA-bisamide, calcium DTPA, and CaNaDTPA-bisamide. Tonicity adjustors useful in a pharmaceutical composition include, without limitation, salts such as, e.g., sodium chloride, potassium chloride, mannitol or glycerin and other pharmaceutically acceptable tonicity adjustor. The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. It is understood that these and other substances known in the art of pharmacology can be included in a pharmaceutical composition.

A therapeutic disclosed herein, or a pharmaceutical composition comprising such a therapeutic compound, may be formulated for either local or systemic delivery using topical, enteral or parenteral routes of administration. Additionally, a therapeutic disclosed herein may be formulated by itself in a pharmaceutical composition, or may be formulated together with one or more other therapeutic compounds disclosed herein in a single pharmaceutical composition or multiple pharmaceutical compositions administered to the individual at the same time or at different times.

A therapeutic disclosed herein, or a pharmaceutical composition comprising such a therapeutic, may be made into an inhaled pharmaceutical formulation. Inhaled pharmaceutical formulations suitable for enteral or parenteral administration include, without limitation, aerosols, dry powders. A therapeutic or a pharmaceutical composition disclosed herein intended for such administration may be prepared according to any method known to the art for the manufacture of pharmaceutical compositions.

In such inhaled dosage forms, the therapeutic may be prepared for delivery as an aerosol in a liquid propellant for use in a pressurised (PDI) or other metered dose inhaler (MDI). Propellants suitable for use in a PDI or MDI include, without limitation, CFC-12, HFA-134a, HFA-227, HCFC-22 (difluorochloromethane), HFA-152 (difluoroethane and isobutane). A therapeutic may also be delivered using a nebulisers or other aerosol delivery system. A therapeutic may be prepared for delivery as a dry powder for use in a dry powder inhaler (DPI). A dry powder for use in the inhalers will usually have a mass median aerodynamic diameter of less than 30 pm, preferably less than 20 pm and more preferably less than 10 pm. Microparticles having aerodynamic diameters in the range of about 5 pm to about 0.5 pm will generally be deposited in the respiratory bronchioles, whereas smaller particles, having aerodynamic diameters in the range of about 2 pm to about 0.05 pm, are likely to be deposited in the alveoli. A DPI may be a passive delivery mechanism, which relies on the individual's inspiration to introduce the particles into the lungs, or an active delivery mechanism, requiring a mechanism for delivering the powder to the individual. In inhalatory pharmaceutical formulations, a therapeutically effective amount of a therapeutic compound disclosed herein for an inhaled formulation may be between about 0.0001% (w/v) to about 60% (w/v), about 0.001% (w/v) to about 40.0% (w/v), or about 0.01% (w/v) to about 20.0% (w/v). In inhalatory pharmaceutical formulations, a therapeutically effective amount of a therapeutic disclosed herein for an inhaled formulation may also be between about 0.0001% (w/w) to about 60% (w/w), about 0.001% (w/w) to about 40.0% (w/w), or about 0.01% (w/w) to about 20.0% (w/w).

A therapeutic disclosed herein, or a pharmaceutical composition comprising such a therapeutic, may be made into a solid pharmaceutical formulation. Solid pharmaceutical formulations suitable for enteral or parenteral administration include, without limitation, capsules, tablets, pills, troches, lozenges, powders and granules suitable for inhalation or for reconstitution into sterile injectable solutions or dispersions. A therapeutic or a pharmaceutical composition disclosed herein intended for such administration may be prepared according to any method known to the art for the manufacture of pharmaceutical compositions. In such solid dosage forms, the therapeutic may be admixed with (a) at least one inert customary excipient (or carrier), such as, e.g., sodium citrate or dicalcium phosphate or (b) fillers or extenders, as for example, starch, lactose, sucrose, glucose, mannitol, isomalt, and silicic acid, (c) binders, such as, e.g., carboxymethylcellulose, alignates, gelatin, polyvinylpyrrolidone, sucrose and acacia, (d) humectants, such as, e.g., glycerol, (e) disintegrating agents, such as, e.g., agar-agar, calcium carbonate, corn starch, potato starch, tapioca starch, alginic acid, certain complex silicates and sodium carbonate, (f) solution retarders, such as, e.g., paraffin, (g) absorption accelerators, such as, e.g., quaternary ammonium compounds, (h) wetting agents, such as, e.g., cetyl alcohol and glycerol monostearate, (i) adsorbents, such as, e.g., kaolin and bentonite, (j) lubricants, such as, e.g., talc, stearic acid, calcium stearate, magnesium stearate, solid polyethylene glycols, sodium lauryl sulfate or mixtures thereof, and (k) buffering agents. The tablets may be uncoated, or they may be coated by known techniques to delay disintegration and absorption in the gastrointestinal tract and thereby provide a sustained action over a longer period. For example, a time delay material such as glyceryl monostearate or glyceryl distearate may be employed. In solid formulations, a therapeutically effective amount of a therapeutic disclosed herein typically may be between about 0.0001% (w/w) to about 60% (w/w), about 0.001% (w/w) to about 40.0% (w/w), or about 0.01% (w/w) to about 20.0% (w/w).

A therapeutic disclosed herein, or a pharmaceutical composition comprising such a therapeutic, may be made into a semi-solid formulation. Semi-solid formulations suitable for topical administration include, without limitation, ointments, creams, salves, and gels. A therapeutic or a pharmaceutical composition disclosed herein intended for such administration may be prepared according to any method known to the art for the manufacture of pharmaceutical compositions. In semi-solid formulations, a therapeutically effective amount of a therapeutic disclosed herein typically may be between about 0.0001% (w/v) to about 60% (w/v), about 0.001% (w/v) to about 40.0% (w/v), or about 0.01% (w/v) to about 20.0% (w/v). In semi-solid formulations, a therapeutically effective amount of a therapeutic disclosed herein typically may also be between about 0.0001% (w/w) to about 60% (w/w), about 0.001% (w/w) to about 40.0% (w/w), or about 0.01% (w/w) to about 20.0% (w/w).

A therapeutic disclosed herein, or a pharmaceutical composition comprising such a therapeutic, may be made into a liquid formulation. Liquid formulations suitable for enteral or parenteral administration include, without limitation, solutions, syrups, elixirs, dispersions, emulsions, and suspensions. A therapeutic or a pharmaceutical composition disclosed herein intended for such administration may be prepared according to any method known to the art for the manufacture of pharmaceutical compositions. In such liquid dosage forms, a therapeutic compound or composition disclosed herein may be admixed with (a) suitable aqueous and nonaqueous carriers, (b) diluents, (c) solvents, such as, e.g., water, ethanol, propylene glycol, polyethyleneglycol, glycerol, vegetable oils, such as, e.g., rapeseed oil and olive oil, and injectable organic esters such as ethyl oleate; and/or fluidity agents, such as, e.g., surfactants or coating agents like lecithin. In the case of dispersions and suspensions, fluidity can also be controlled by maintaining a particular particle size. In liquid formulations, a therapeutically effective amount of a therapeutic disclosed herein typically may be between about 0.0001% (w/v) to about 60% (w/v), about 0.001% (w/v) to about 40.0% (w/v), or about 0.01% (w/v) to about 20.0% (w/v).

Syrups and elixirs may be formulated with sweetening agents, for example glycerol, propylene glycol, sorbitol or sucrose. Such formulations may also contain a demulcent, a preservative, flavoring agents, and coloring agents.

Liquid suspensions may be formulated by suspending a therapeutic disclosed herein in admixture with excipients suitable for the manufacture of aqueous suspensions. Such excipients are suspending agents, for example sodium carboxymethylcellulose, methylcellulose, hydroxypropylmethylcellulose, sodium alginate, pectin, polyvinyl pyrrolidone, polyvinyl alcohol, natural gum, agar, gum tragacanth and gum acacia; dispersing or wetting agents may be a naturally occurring phosphatide, for example lecithin, or condensation products of an alkylene oxide with fatty acids, for example polyoxyethylene stearate, or condensation products of ethylene oxide with long-chain aliphatic alcohols, for example heptadecaethyleneoxycetanol, or condensation products of ethylene oxide with partial esters derived from fatty acids, for example polyoxyethylene sorbitan monooleate.

Oily suspensions may be formulated by suspending a therapeutic disclosed herein in admixture with (a) vegetable oils, such as, e.g., almond oil, arachis oil, avocado oil, canola oil, castor oil, coconut oil, corn oil, cottonseed oil, grape seed oil, hazelnut oil, hemp oil, linseed oil, olive oil, palm oil, peanut oil, rapeseed oil, rice bran oil, safflower oil, sesame oil, soybean oil, soya oil, sunflower oil, walnut oil, wheat germ oil, or a combination thereof, (b) a saturated fatty acid, an unsaturated fatty acid, or a combination thereof, such as, e.g., palmitic acid, stearic acid, oleic acid, linoleic acid, linolenic acid, or a combination thereof, (c) mineral oil such as, e.g., liquid paraffin, (d) surfactants or detergents. The oily suspensions may contain a thickening agent, for example beeswax, hard paraffin or cetyl alcohol. Sweetening agents, such as those set forth above, and flavoring agents may be added to provide a palatable oral preparation. These pharmaceutical compositions may be preserved by the addition of an antioxidant such as ascorbic acid.

Dispersible powders and granules suitable for preparation of an aqueous suspension by the addition of water provide the combined therapeutic in admixture with a dispersing or wetting agent, suspending agent and one or more preservatives.

A therapeutic disclosed herein may be in the form of oil-in-water emulsions. The oily phase may be a vegetable oil as disclosed herein or a mineral oil as disclosed herein or mixtures thereof. Suitable emulsifying agents may be naturally occurring gums, such as, e.g., gum acacia or gum tragacanth, naturally occurring phosphatides, for example soya bean, lecithin, and esters or partial esters derived from fatty acids and hexitol anhydrides, for example sorbitan monooleate and condensation products of the said partial esters with ethylene oxide, for example polyoxyethylene sorbitan monooleate.

A therapeutic disclosed herein, or a pharmaceutical composition comprising such a therapeutic, may also be incorporated into a drug delivery platform in order to achieve a controlled release profile over time. Such a drug delivery platform comprises a therapeutic disclosed herein dispersed within a polymer matrix, typically a biodegradable, bioerodible, and/or bioresorbable polymer matrix. As used herein, the term “polymer” refers to synthetic homo- or copolymers, naturally occurring homo- or copolymers, as well as synthetic modifications or derivatives thereof having a linear, branched or star structure. Copolymers can be arranged in any form, such as, e.g., random, block, segmented, tapered blocks, graft, or triblock. Polymers are generally condensation polymers. Polymers can be further modified to enhance their mechanical or degradation properties by introducing cross-linking agents or changing the hydrophobicity of the side residues. If crosslinked, polymers are usually less than 5% crosslinked, usually less than 1% crosslinked.

Suitable polymers include, without limitation, alginates, aliphatic polyesters, polyalkylene oxalates, polyamides, polyamidoesters, polyanhydrides, polycarbonates, polyesters, polyethylene glycol, polyhydroxyaliphatic carboxylic acids, polyorthoesters, polyoxaesters, polypeptides, polyphosphazenes, polysaccharides, and polyurethanes. The polymer usually comprises at least about 10% (w/w), at least about 20% (w/w), at least about 30% (w/w), at least about 40% (w/w), at least about 50% (w/w), at least about 60% (w/w), at least about 70% (w/w), at least about 80% (w/w), or at least about 90% (w/w) of the drug delivery platform. Examples of biodegradable, bioerodible, and/or bioresorbable polymers and methods useful to make a drug delivery platform are described in, e.g., Drost, et. al., Controlled Release Formulation, U.S. Pat. No. 4,756,911; Smith, et. al., Sustained Release Drug Delivery Devices, U.S. Pat. No. 5,378,475; Wong and Kochinke, Formulation for Controlled Release of Drugs by Combining Hyrophilic and Hydrophobic Agents, U.S. Pat. No. 7,048,946; Hughes, et. al., Compositions and Methods for Localized Therapy of the Eye, U.S. Patent Publication 2005/0181017; Hughes, Hypotensive Lipid-Containing Biodegradable Intraocular Implants and Related Methods, U.S. Patent Publication 2005/0244464; Altman, et al., Silk Fibroin Hydrogels and Uses Thereof, U.S. Patent Publication 2011/0008437; each of which is incorporated by reference in its entirety.

In aspects of this embodiment, a polymer composing the matrix is a polypeptide such as, e.g., silk fibroin, keratin, or collagen. In other aspects of this embodiment, a polymer composing the matrix is a polysaccharide such as, e.g., cellulose, agarose, elastin, chitosan, chitin, or a glycosaminoglycan like chondroitin sulfate, dermatan sulfate, keratan sulfate, or hyaluronic acid. In yet other aspects of this embodiment, a polymer composing the matrix is a polyester such as, e.g., D-lactic acid, L-lactic acid, racemic lactic acid, glycolic acid, caprolactone, and combinations thereof.

One of ordinary skill in the art appreciates that the selection of a suitable polymer for forming a suitable disclosed drug delivery platform depends on several factors. The more relevant factors in the selection of the appropriate polymer(s), include, without limitation, compatibility of polymer with drug, desired release kinetics of drug, desired biodegradation kinetics of platform at implantation site, desired bioerodible kinetics of platform at implantation site, desired bioresorbable kinetics of platform at implantation site, in vivo mechanical performance of platform, processing temperatures, biocompatibility of platform, and patient tolerance. Other relevant factors that, to some extent, dictate the in vitro and in vivo behavior of the polymer include the chemical composition, spatial distribution of the constituents, the molecular weight of the polymer and the degree of crystallinity.

A drug delivery platform includes both a sustained release drug delivery platform and an extended release drug delivery platform. As used herein, the term “sustained release” refers to the release of a therapeutic compound disclosed herein over a period of about seven days or more. As used herein, the term “extended release” refers to the release of a therapeutic disclosed herein over a period of time of less than about seven days.

In aspects of this embodiment, a sustained release drug delivery platform releases a therapeutic disclosed herein with substantially zero order release kinetics over a period of, e.g., about 7 days after administration, about 15 days after administration, about 30 days after administration, about 45 days after administration, about 60 days after administration, about 75 days after administration, or about 90 days after administration. In other aspects of this embodiment, a sustained release drug delivery platform releases a therapeutic disclosed herein with substantially zero order release kinetics over a period of, e.g., at least 7 days after administration, at least 15 days after administration, at least 30 days after administration, at least 45 days after administration, at least 60 days after administration, at least 75 days after administration, or at least 90 days after administration.

In aspects of this embodiment, a sustained release drug delivery platform releases a therapeutic disclosed herein with substantially first order release kinetics over a period of, e.g., about 7 days after administration, about 15 days after administration, about 30 days after administration, about 45 days after administration, about 60 days after administration, about 75 days after administration, or about 90 days after administration. In other aspects of this embodiment, a sustained release drug delivery platform releases a therapeutic disclosed herein with substantially first order release kinetics over a period of, e.g., at least 7 days after administration, at least 15 days after administration, at least 30 days after administration, at least 45 days after administration, at least 60 days after administration, at least 75 days after administration, or at least 90 days after administration.

In aspects of this embodiment, a drug delivery platform releases a therapeutic compound disclosed herein with substantially zero order release kinetics over a period of, e.g., about 1 day after administration, about 2 days after administration, about 3 days after administration, about 4 days after administration, about 5 days after administration, or about 6 days after administration. In other aspects of this embodiment, a drug delivery platform releases a therapeutic disclosed herein with substantially zero order release kinetics over a period of, e.g., at most 1 day after administration, at most 2 days after administration, at most 3 days after administration, at most 4 days after administration, at most 5 days after administration, or at most 6 days after administration.

In aspects of this embodiment, a drug delivery platform releases a therapeutic disclosed herein with substantially first order release kinetics over a period of, e.g., about 1 day after administration, about 2 days after administration, about 3 days after administration, about 4 days after administration, about 5 days after administration, or about 6 days after administration. In other aspects of this embodiment, a drug delivery platform releases a compound disclosed herein with substantially first order release kinetics over a period of, e.g., at most 1 day after administration, at most 2 days after administration, at most 3 days after administration, at most 4 days after administration, at most 5 days after administration, or at most 6 days after administration.

Wide variations in the necessary effective amount are to be expected in view of the differing efficiencies of the various routes of administration. For instance, oral administration of a therapeutic\disclosed herein generally would be expected to require higher dosage levels than administration by inhalation. Similarly, systemic administration of a therapeutic disclosed herein would be expected to require higher dosage levels than a local administration. Variations in these dosage levels can be adjusted using standard empirical routines of optimization, which are well-known to a person of ordinary skill in the art. The precise therapeutically effective dosage levels and patterns are preferably determined by the attending physician in consideration of the above-identified factors. One skilled in the art will recognize that the condition of the individual can be monitored throughout the course of therapy and that the effective amount of a therapeutic compound disclosed herein that is administered can be adjusted accordingly.

### EXAMPLES

The following non-limiting examples are provided for illustrative purposes only in order to facilitate a more complete understanding of representative embodiments now contemplated. These examples are intended to be a mere subset of all possible contexts in which the components of the formulation may be combined. Thus, these examples should not be construed to limit any of the embodiments described in the present specification, including those pertaining to the type and amounts of components of the formulation and/or methods and uses thereof. As used in the below examples, the term “SA inhibitor” refers to an isolated peptide with the sequence Ac-Pro-Pro-Pro-His-Pro-His-Ala-Arg-Ile-Lys-NH₂ (SEQ ID NO: 2).

### Example 1

### Androgen-Regulation of TMPRSS2 and ACE2 in LNCaP and A549 Cells

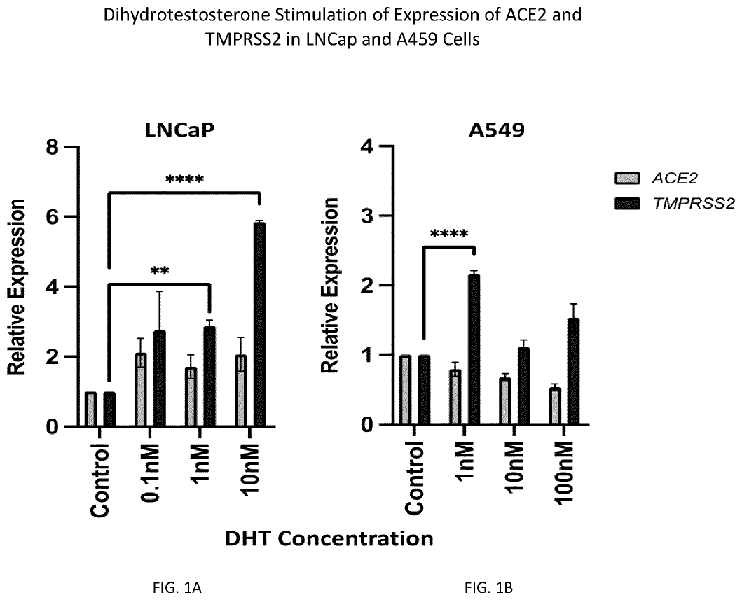

LNCaP (prostate) and A549 (Androgen receptor positive, human alveolar carcinoma-derived cell line with type II pneumonocyte properties) cells were seeded in 12-well plates (2×10⁵ cells per well) in phenol red free RPMI-1640 and DMEM media (Lonza), supplemented with 5% and 2% double charcoal stripped FCS (First Link UK) respectively, and 2 mM I-glutamine, 100 units/ml penicillin and 100 mg/ml streptomycin (Sigma). Charcoal stripping was required to remove residual traces of hormones (Sedelaar et al, (2009)). Cells were treated with 0-100 nM dihydrotestosterone (DHT, Sigma) for 6 hrs. RNA was extracted using TRIzol (Thermo Fisher Scientific) and reverse transcribed (LunaScript RT Supermix Kit, NEB). TMPRSS2 and ACE2 expression was quantified using qPCR (LightCycler 96, Roche) and the data normalized to an internal control (L19). The mean of three independent repeats ±1SE are shown in FIG. 1A and FIG. 1B. ANOVA, **=p<0.01, ****=p<0.0001

As shown in FIG. 1A, the expression of TMPRSS2, but not ACE2, showed a dose response to stimulation of LNCaP cells with dihydrotestosterone with the relative expression at 0.1 nM approximately 3× compared to cells grown in the absence of dihydrotestosterone. The relative expression of TMPRSS2 increased to 6× at 10 nM dihydrotestosterone compared to the control.

As shown in FIG. 1B, in A549 type ii pneumonocytes, a target cell type for SARS-COV-2, TMPRSS2 increased by over 2× with 1 nM dihydrotestosterone compared to cells grown in the absence of dihydrotestosterone. The relative expression of ACE2 demonstrated a slight negative correlation with dihydrotestosterone concentration.

### Example 2

### SA Inhibitor Suppression of TMPRSS2 Levels in A549 in Full Media

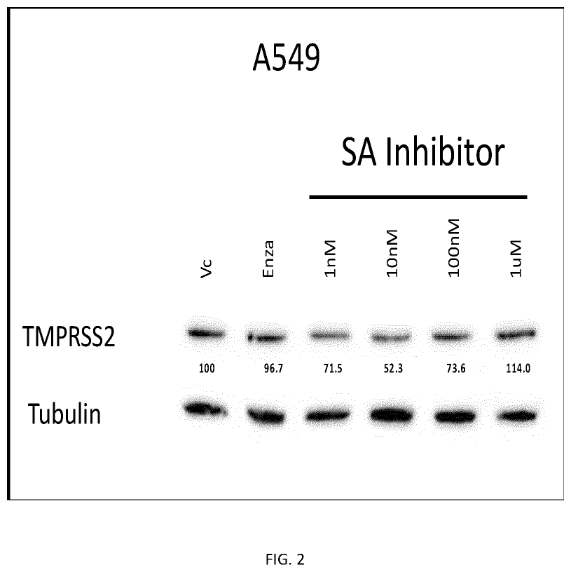

A549 were seeded in 6-well plates at a density of 1×10⁶ per well. Cells were incubated in DMEM media (Lonza), supplemented with 10% FCS (HyClone) and 2 mM !-glutamine, 100 units/ml penicillin and 100 mg/ml streptomycin (Sigma). Fetal calf serum has been shown to contain castrate levels of testosterone, which LNCaP cells metabolise to produce physiologically relevant intracellular levels of dihydrotestosterone, sufficient to promote their growth. Cells were treated with 0-1 μM SA Inhibitor (SEQ ID NO: 2) or 10 μM Enzalutamide (ENZA, Sigma) for 72 hrs. Cells were harvested and proteins separated using SDS-PAGE. Blots were probed with antibodies specific for TMPRSS2 (ab92323, Abcam) and α-tubulin (B-5-1-2, Sigma). Protein levels were visualized using a chemiluminescence imaging system (Fusion FX, Vilber). Densitometry was performed using Image J and TMPRSS2 levels normalized to the internal control (Tubulin). The expression of TMPRSS2 protein, shown in FIG. 2, exhibited a U-shaped response to of SA Inhibitor with decreased expression up to 10 nM SA Inhibitor followed by a progressive increase up to 1 μM. This confirms that Scr Kinase-Androgen Receptor blockade is capable of down regulating TMPRSS2 expression in lung cells. By comparison, enzalutamide, a classic androgen receptor antagonist did not achieve down regulation.

### Example 3

### SA Inhibitor Suppression of TMPRSS2 Levels in A549 in Full Media

LNCaP and A549 cells were seeded at a density of 1×10⁶ in 6-well plates and incubated for 72 hours in phenol red free RPM1-1640 and DMEM media (Lonza), supplemented with 5% and 2% double charcoal stripped FCS (First Link UK) respectively, and 2 mM I-glutamine, 100 units/ml penicillin and 100 mg/ml streptomycin (Sigma). Cells were treated with 100 ng/ml Epithelial Growth Factor (EGF, Sigma) or 1 nM DHT (dihydrotestosterone, Sigma) ±SA Inhibitor for 72 hrs. Cells were harvested and proteins separated using SDS-PAGE. Blots were probed with antibodies specific for TMPRSS2 (ab92323, Abcam) and α-tubulin (B-5-1-2, Sigma). Protein levels were visualized using a chemiluminescence imaging system (Fusion FX, Vilber). Densitometry was performed using Image J and TMPRSS2 levels normalised to the internal control (Tubulin).

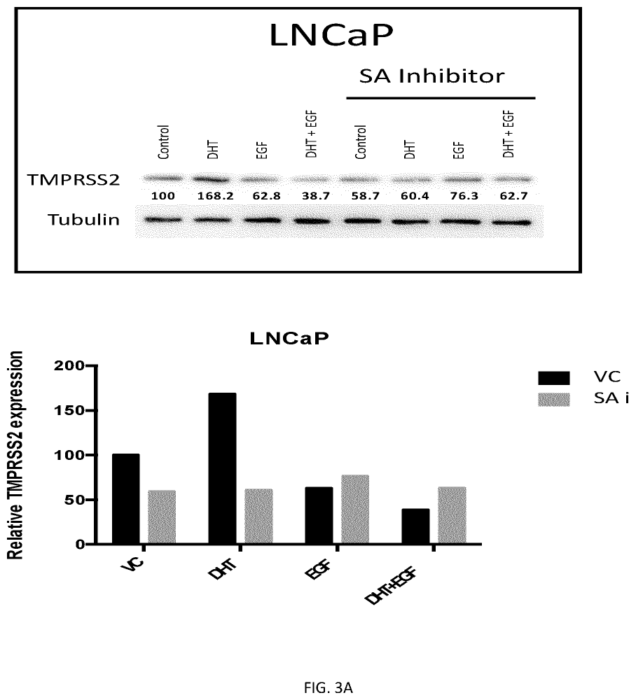

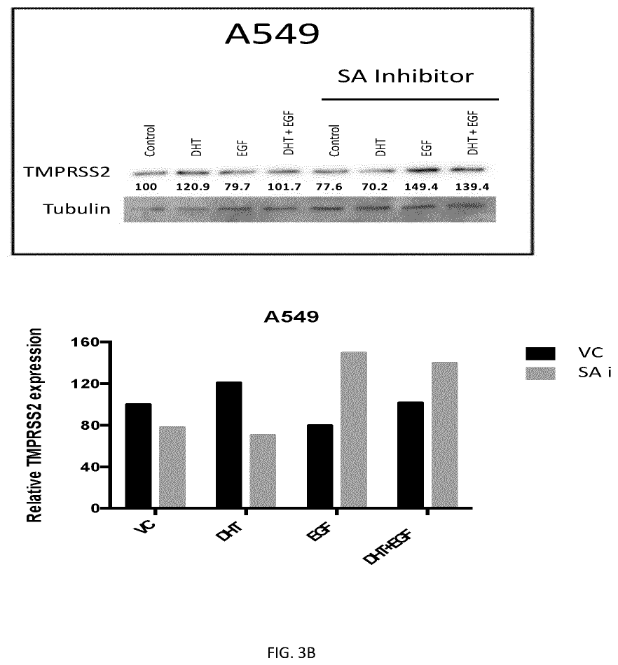

The results shown in FIG. 3A and FIG. 3B demonstrate TMPRSS2 expression is upregulated by dihydrotestosterone (relative expression 168% in LNCaP and 120.9% in A549 cells) and down regulated by EGF (relative expression 62.8% in LNCaP and 797% in A549 cells). Combined dihydrotestosterone and Epithelial Growth Factor stimulation down regulated TMPRSS2 expression in LNCAP cells and to a lesser extent in A459 cells. SA Inhibitor reduced TMPRSS2 protein expression to levels below the non-DHT stimulated control both in the absence of DHT stimulation (LNCaP 58.7% and A549 77.6%) and, importantly, with DHT stimulation (LNCaP 60.4% and A549s 70.2%). The SA Inhibitor clearly reversed the stimulatory effects of dihydrotestosterone on TMPRSS2 in both cell lines. The magnitude of the response was consistent with previously reported Androgen receptor levels within the prostate derived (higher) and lung derived (lower) cell lines and supports the therapeutic use of SA Inhibitor to reduce TMPRSS2 mediated Viral cell entry. EGF suppressed TMPRSS2 expression both alone and in combination with DHT at the concentrations indicated, consistent with literature reports (Mikkonen et al, (2010)). SA inhibitor reversed this inhibitory effect indicating a common mechanistic pathway.

### Example 4

### SA Inhibitor Suppression of NETosis Quantified by Nucleosome Release

The ability of SA Inhibitor to reduce NETosis in Neutrophils stimulated by lipopolysaccharide (LPS, Sigma Aldrich, Product Number L4391-1MG) alone or in combination with Epidermal growth factor (Sigma Aldrich, Product Number SRP3027-500UG) was compared to the efficacy of a Src kinase Inhibitor (PP2, Sigma Aldrich, Product Number P0042-5MG) and a PAD4 inhibitor (GSK484, Cambridge Bioscience UK, Product Number CAY17488-1 mg) which is known to down regulate NETosis by preventing citrullination of histone proteins.

Whole blood was collected from healthy male volunteers (100-150 mL per donor using K2-EDTA anti-coagulant (Bioscience, UK). Blood was transported at ambient temperature (˜21° C.) overnight and neutrophils isolated within 24 hrs of collection with EasySep™ direct human Neutrophil Isolation kits (Stemcell™ Technologies, Product Number 19666) according to the manufacturer's instructions. Blood samples, Phosphate Buffered Saline with 1 mM EDTA (UltraPure™ 0.5 M EDTA, pH 8.0 Invitrogen, Fisher, Product Number 11568896) and centrifuge were brought to room temperature (RT ˜21° C.) and the NET assay buffer comprising Phenol Red Free RPMI-1640 media (Thermo-Fisher, Product Number 11835030)+3% Charcoal stripped FBS (Gibco™ Fisher, Product Number, 15634559)+1% Bovine Serum Albumin was pre-warmed to 37° C. in water bath. 25 ml whole blood was added to a 50 ml conical falcon tube (Tube-1) and 1250 μL Isolation Cocktail added. RapidSpheres™ were vortexed for 30 seconds to re suspend and 1250 μL added to Tube-1. The sample and beads were mixed by gently pipetting and incubated at RT for 5 minutes. 22 ml of Neutrophil isolation medium was added to the sample mixed by gently pipetting up and down 3 times. The tube (without lid) was placed into the EasySep™ magnet and following incubation at RT for 10 minutes the entire clear top fraction carefully collected using a single pipette and transferred to a fresh 50 ml conical falcon tube (Tube-2). A second aliquot of RapidSpheres™ was vortexed for 5 seconds and 1250 μL added to Tube-2) containing the enriched cells and mixed by gently pipetting followed by incubated at RT for 5 minutes.

Tube-2 was placed into the magnet and after 10 minutes incubation the clear fraction (enriched cell suspension) collected by single pipette and transferred into a fresh 50 ml conical falcon tube (Tube-3) which was placed back into the magnet for a final, third separation. The clear fraction, containing purified, isolated neutrophils was transferred into a fresh 50 ml conical falcon tube (Tube-4) and the neutrophil cell Number, % viability and yield determined by cell counting and Trypan blue exclusion.

During the neutrophil isolation procedure, intermediate dilutions of the SA Inhibitor, PP2 or GSK484 or DMSO vehicle (at 20× to final concentration required) from 10 mM stocks in NET assay buffer (pre-warmed to 37° C. in water bath) as shown in table 1:
SA Inhibitor (ValiRx Plc, VAL201): 10 mM stocks were made in deionized water
SrcKi (PP2): 10 mM stocks were made in DMSO
PADi4 (GSK484): 10 mM stocks were made in DMSO

TABLE 1
Inhibitor stock, intermediate and working concentrations

| | Stock conc. | Final conc. required | Prepare at 20X |
|---|---|---|---|
| Inhibitor | (mM) | (mM) (1X) | conc. (mM) |
| SA I | 10 | 10 | 200 |
| (VAL201) | | 1 | 20 |
| | | 0.1 | 2 |
| SrcKi (PP2) | 10 | 10 | 200 |
| | | 1 | 20 |
| | | 0.1 | 2 |
| PADi4 | 10 | 10 | 200 |
| (GSK484) | | 1 | 20 |
| | | 0.1 | 2 |

The purified Neutrophil cell suspension was pelleted by centrifuge and resuspended in the NET assay buffer (pre-warmed to 37° C. water bath) at a concentration of ˜1.11×10⁶/mL. 50 μL of intermediate inhibitor dilutions prepared according to the table above or DMSO vehicle were added to individual wells of a 24-well plate as shown in the in table 2.

TABLE 2
Plate map showing inhibitor working concentrations.

| | 1 | 2 | 3 | 4 | 5 | 6 |
|---|---|---|---|---|---|---|
| A | Buffer | Buffer | Buffer | Buffer | Buffer | Buffer |
| | (1% DMSO) | (1% DMSO) | (1% DMSO) | (1% DMSO) | (1% DMSO) | (1% DMSO) |
| B | SA I - 0.1 uM | SA I - 0.1 uM | PP2 - 0.1 uM | PP2 - 0.1 uM | GSK484 - 0.1 uM | GSK484 - 0.1 uM |
| C | SA I - 1.0 uM | SA I - 1.0 uM | PP2 - 1.0 uM | PP2 - 1.0 uM | GSK484 - 1.0 uM | GSK484 - 1.0 uM |
| D | SA I - 1.0 uM | SA I - 1.0 uM | PP2 - 10 uM | PP2 - 10 uM | GSK484 - 10 uM | GSK484 - 10 uM |

900 uL of the cell suspension (1×10⁶/mL cells/well) was added to each well of above 24-well plate and briefly mixed on a plate shaker at 300 rpm for 30 seconds. The inhibitors were pre-incubated with the cells for 30 min in a humidified 37° C., 5% CO₂ incubator. During this period, intermediate activator dilutions were prepared from stock solutions at 20× final concentration in the NET assay buffer and prewarmed to 37° C. in a water bath.

During the pre-incubation with SA inhibitor or positive controls, intermediate solutions of Lipopolysaccharide (NETosis trigger) and Epidermal Growth Factor were prepared to 20× final required concentration in NET assay buffer prewarmed to 37° C. as indicated in table 3.

TABLE 3
NETosis trigger stock, intermediate and working concentrations.

| | Stock conc. | Final conc. Required | Intermediate 20x |
|---|---|---|---|
| Compound | (ug/mL) | (ng/mL) | conc. (ng/mL) |
| LPS | 1000 | 2000 | 40,000 |
| EGF | 500 | 10 | 200 |

Following the 30 minute preincubation of the Neutrophils with SA inhibitor or control compounds, NETosis was stimulated by addition of 50 uL of 20× stock solution of LPS or 50 uL each of LPS and EGF and mixed for 30 seconds on an orbital shaker (300 rpm).

After four hours stimulation in a humidified 37° C., 5% CO₂ incubator 10 μl DNAse solution (Fisher, Product Number 10636153) at 1010 U/ml, was added to the Neutrophil assay plate (10U/ml final concentration) and incubated for 5 minutes at room temperature (˜21° C.) on a plate shaker plate shaker at 300 rpm. At the end of this 5-minute step, to release externalised NETs from the Neutrophils, 20 μl of 500 mM EDTA solution was added, and the plate mixed for 30 seconds on a plate shaker (300 rpm) to inactivate the DNAse. The Neutrophil assay plates were centrifuged at 4000 rpm for 10 minutes on a plate centrifuge to pellet cells and the supernatant (˜950 μI) carefully collected from each well without disturbing the pellet and transferred to a low bind, 96-deep well plate. The supernatant plates were stored at −80° C. until analysis.

The level of NETs released into the supernatant were quantified by automated chemiluminescence immunoassay (IDS-i10 Immunoanalyzer, Immunodiagnostics Systems Ltd) targeting nucleosomes (including oligonucleosomes) containing histone 3.1 variant or histone 3 citrullinated at the arginine 8 position. For the H3.1 nucleosome immunoassay, 3 uL of cell supernatant containing released NETs from each condition was diluted into 137 uL Assay buffer (200 mM Phosphate Buffered Saline containing 500 ug HAMA blocker). For the H3R8 citrullinated nucleosomes 50 uL of cell supernatant containing released NETs from each condition was diluted into 100 uL Assay buffer. The diluted supernatants were incubated with 50 uL of acridinium ester conjugated anti-nucleosomes antibody for 30 minutes followed by addition of 20 uL MyOne™ Tosylated beads coated with antibodies directed towards the H3.1 or citrullinated H3R8 residue of the cell free nucleosomes and a further 15-minute incubation. The magnetic beads were then washed, trigger solution added followed and the flash chemiluminescence signal read as Relative Light Units. The results are reported as the mean of two replicates with coefficient of variance between the replicates displayed.

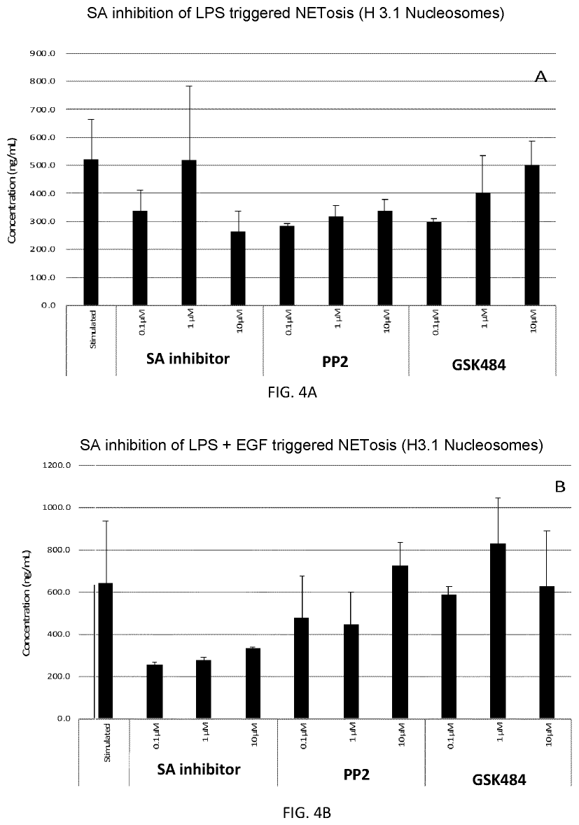

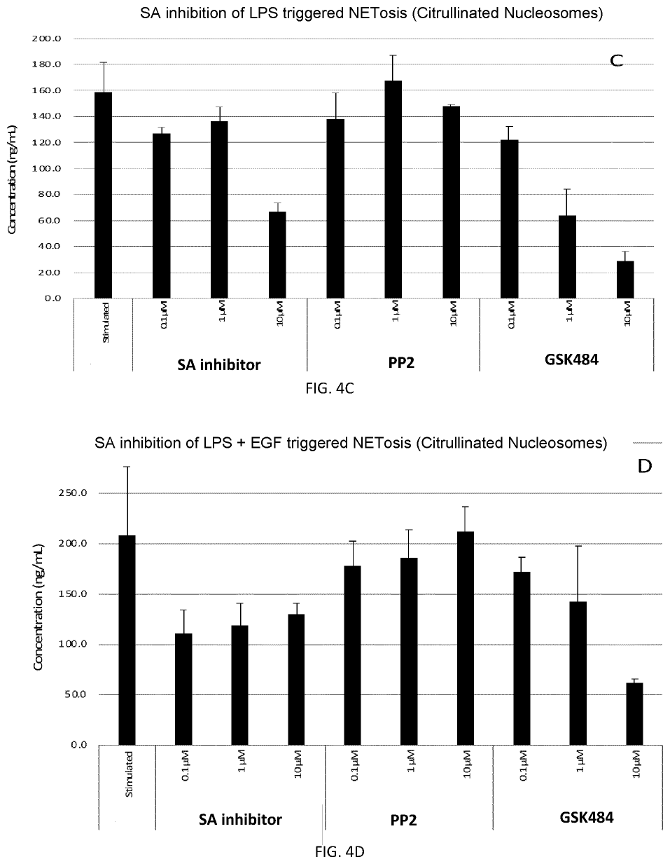

Cell free nucleosomes levels are a surrogate marker of NETosis with citrullinated nucleosomes more specifically associated with NETs and their degradation products. The results shown in FIG. 4A show that 0.1 uM SA Inhibitor inhibited LPS triggered NETosis, determined through release of H3.1 nucleosomes, from an average of 523.2 ng/mL to 339.4 ng/mL. FIG. 4B shows that LPS in combination with EGF triggered a higher release (641.9 ng/mL) with even more efficient suppression of H3.1 NETosis derived nucleosome levels to 254.2 ng/mL. FIG. 4 C shows a marked reduction in levels of citrullinated nucleosomes, from an average of 158 ng/mL triggered by LPS, to 126.5 ng/mL with 0.1 uM SA Inhibitor. A further decrease to 66.6 ng/mL was achieved at 10 uM SA Inhibitor. FIG. 4D shows combined LPS and EGF stimulation of NETosis resulted in 208.5 ng/mL of citrullinated nucleosomes which was reduced to 111.1 ng/mL with 0.1 uM SA Inhibitor. FIG. 4 C and FIG. 4D also clearly show that PP2, a general Src Kinase Inhibitor was ineffective at suppressing NETosis determined with the more specific citrullinated nucleosome marker whereas the PAD4i, citrullination inhibitor showed a dose response reduction in citrullinated nucleosomes levels as expected.

### Example 5

### Live Cell Imaging and Quantification of Androgen Triggered NETosis Inhibition

Neutrophils, isolated as described in Example 4 (or by equivalent methodology as known in the art, for example, Ficoll Density gradient and dextran sedimentation followed by Red blood cell lysis with hypertonic solution) are resuspended in phenol red free RPMI 1640 media at a density of 2×10⁶ cells per milliliter and incubated for five minutes in the dark at room temperature with NUCLEAR-ID Red (membrane impermeable) DNA dye to stain nuclei (1 ml/1.5 ml of cell suspension; Enzo Life Sciences). Cells are washed three times to remove free Dye by centrifuging at 2400 RFI for 5 min to pellet the cells and replacing supernatant with 1 ml fresh phenol red free RPMI 1640 media. After the final wash step, Incucyte® Cytotox Green Dye is diluted to a final assay concentration of 250 nM in serum free, phenol red free RPMI 1640 media and used to prepare a neutrophil stock at a density of 200,000 cells/mL.

Neutrophils in media containing Cytotox Green Dye are then plated into a clear bottom 96 well plate pre-coated with 50 uL/well Fibronectin (1 mg/mL stock, Sigma Cat. No. F1141) at 20,000 neutrophils per 100 ul/well in phenol red free RMPI media. 4× concentrated SA Inhibitor dilutions are prepared from a stock solution (10 mM in PBS) by dilution in phenol red free RPMI 1640 media sufficient to provide a final working range of 1 nM-1 mM. Following a control condition with no SA inhibitor consisting of 50 uL phenol red free RPMI media, 50 uL of each SA Inhibitor is added running down the plate in triplicates and incubated at 37° C. in 5% CO2 for 30 minutes. 4× concentrated Dihydrotestosterone dilutions (DHT, Sigma) are prepared from by dilution in phenol red free RPMI 1640 media containing 80 ug LPS NETosis trigger (4× working concentration) sufficient to generate a final working range of 0.1 nm, 1 nm or 10 nm DHT. 50 uL of each LPS solution and a negative DHT control) is added to the triplicates across the plate to generate a checkerboard of SA inhibitor and DHT activator with a final concentration of 20 ug/mL LPS trigger as shown in Table 4.

TABLE 4
Plate map showing SA inhibitor (SA I) and DHT activator concentrations for live cell imaging

| | DHT Concentration at an LPS trigger concentration of 20 ug/mL | | | | | | | | | | | | | | | | | | | | | | | |
|---|---|---|---|---|---|---|---|---|---|---|---|---|---|---|---|---|---|---|---|---|---|---|---|---|
| | 0 | | | | | | 0.1 nM DHT | | | | | | 1 nM DHT | | | | | | 10 nM DHT | | | | | |
| SA I | 0 | | 0 | | 0 | | 0 | | 0 | | 0 | | 0 | | 0 | | 0 | | 0 | | 0 | | 0 | |
| concentration | 1 | nM | 1 | nM | 1 | nM | 1 | nM | 1 | nM | 1 | nM | 1 | nM | 1 | nM | 1 | nM | 1 | nM | 1 | nM | 1 | nM |
| | 10 | nM | 10 | nM | 10 | nM | 10 | nM | 10 | nM | 10 | nM | 10 | nM | 10 | nM | 10 | nM | 10 | nM | 10 | nM | 10 | nM |
| | 10² | nM | 10² | nM | 10² | nM | 10² | nM | 10² | nM | 10² | nM | 10² | nM | 10² | nM | 10² | nM | 10² | nM | 10² | nM | 10² | nM |
| | 10³ | nM | 10³ | nM | 10³ | nM | 10³ | nM | 10³ | nM | 10³ | nM | 10³ | nM | 10³ | nM | 10³ | nM | 10³ | nM | 10³ | nM | 10³ | nM |
| | 10⁴ | nM | 10⁴ | nM | 10⁴ | nM | 10⁴ | nM | 10⁴ | nM | 10⁴ | nM | 10⁴ | nM | 10⁴ | nM | 10⁴ | nM | 10⁴ | nM | 10⁴ | nM | 10⁴ | nM |
| | 10⁵ | nM | 10⁵ | nM | 10⁵ | nM | 10⁵ | nM | 10⁵ | nM | 10⁵ | nM | 10⁵ | nM | 10⁵ | nM | 10⁵ | nM | 10⁵ | nM | 10⁵ | nM | 10⁵ | nM |
| | 10⁶ | nM | 10⁶ | nM | 10⁶ | nM | 10⁶ | nM | 10⁶ | nM | 10⁶ | nM | 10⁶ | nM | 10⁶ | nM | 10⁶ | nM | 10⁶ | nM | 10⁶ | nM | 10⁶ | nM |

Neutrophils are imaged within 10 min of plating using phase contrast, red (800-ms exposure), and green (400-ms exposure) channels in an IncuCyte ZOOM platform, housed inside an incubator at 37° C. with 5% CO₂. Three image sets from distinct regions per well are taken every 5-15 min using a 20× dry objective. Representative images of the unstimulated and LPS/DHT stimulated neutrophils are selected and used to train the IncuCyte Basic Software to identify neutrophils through the red stained nuclei and NETosing neutrophils based on the green staining of externalised DNA as described in the literature (e.g. Gupta et al, 2016)). The Number of neutrophils is then calculated as the average red count for the three images per well and the Number of green cells calculated as the average green count. The percentage of neutrophils undergoing NETosis is calculated as the average green count divide by the average red count at each time point measured. The level of NETosis is increased with increasing concentrations of dihydrotestosterone confirming the activation of NETosis through androgen mediated-Androgen Receptor (AR) signalling in neutrophils. This increase is reversed by SA Inhibition confirming the involvement of AR-Src-Kinase activation in NETosis and the therapeutic validity of down regulating this pathway to reduce NETosis.

### Example 6

### Live Cell Imaging and Quantification of IL6 Triggered NETosis Inhibition

In a similar experiment to that described in Example 5, IL6 (Merck, Product Number H7416-10UG) is used to activate NETosis in response to LPS using a similar checkerboard configuration as shown in Table 4 (working IL6 concentrations 0, 10, 50 and 100 ng/mL). The level of NETosis is increased with increasing concentrations of IL6 confirming the activation of NETosis through IL6 mediated—transactivation of Androgen Receptor (AR) signalling in neutrophils. This increase is reversed by SA Inhibition confirming the involvement of AR-Src-Kinase activation in NETosis and the therapeutic validity of down regulating this pathway to reduce NETosis.

### Example 7

### Live Cell Imaging and Quantification of IL8 Triggered NETosis Inhibition

In a similar experiment to that described in Example 5, IL8 (Merck, Product Number 11645-10UG) is used to activate NETosis in response to LPS using a similar checkerboard configuration as shown in Table 4 (working IL6 concentrations 0, 1, 10 and 100 nM). The level of NETosis is increased with increasing concentrations of IL8 confirming the activation of NETosis through IL8 mediated—transactivation of Androgen Receptor (AR) signalling in neutrophils. This increase is reversed by SA Inhibition confirming the involvement of AR-Src-Kinase activation in NETosis and the therapeutic validity of down regulating this pathway to reduce NETosis.

### Example 8

### Live Cell Imaging and Quantification of NETosis Inhibition in COVID-19 Patient Derived Neutrophils

In a similar experiment to that described in Example 5 fresh neutrophils are isolated from a 54-year-old male patient with PCR confirmed SARSCoV-2 and admitted to intensive care for mechanical oxygen support. The isolated neutrophils are treated with SA-Inhibitor and the level of NETosis shown to decrease in the treated neutrophils. This increase is reversed by SA Inhibition confirming the involvement of AR-Src-Kinase activation in NETosis in viral sepsis and the therapeutic validity of down regulating this pathway to reduce NETosis.

### Example 9

SA Inhibitor in Combination with DNASe Treatment for Viral ARDS Using Dry Powder Formulation

A male patient, age 69, with a comorbidity of cardiovascular disease and diabetes is seen in the Emergency Room. The patient is running a temperature of 102° F. and is suffering from ARDS with severe flu like symptoms. PCR testing confirmed the presence of a COVID-19 virus infection. The patient is administered 1 mg/kg of a peptide with a sequence of Ac-Pro-Pro-Pro-His-Pro-His-Ala-Arg-Ile-Lys-NH₂ (SEQ ID NO: 2) co-formulated with 2.5 mg recombinant Dornase Alpha once daily by dry powder inhaler. After a few days of administering the peptide/DNAse combination, the viral load, as measured by PCR, is reduced and lower than would be expected in the absence of the peptide. Additionally, the patient is found to have improved lung function with a higher measured SpO2 level resulting in a lower level of respiratory distress. Reduction of viral load persists for the term of the treatment and treatment is continued until the patient is confirmed to be virus free by PCR. The peptide is continued to be administered to the patient until the patient is able to maintain a normal SpO2 levels for seven days and is found to have a clear chest X-ray.

### Example 10

### SA Inhibitor for Treatment of Bacterial Infection and In-Vivo NETosis

A male patient, age 62, is seen in the Emergency Room. The patient is running a temperature of 102° F. with a heart rate exceeding 90 beats/minute and rapid breathing. Septic shock is confirmed based on the requirement for vasopresser administration to maintain a mean arterial pressure of 65 mmHg and serum lactate level over 2 mmol/L. The patient is noted to be suffering from impaired kidney function. The patient is administered 4 mg/kg of a peptide with a sequence of Ac-Pro-Pro-Pro-His-Pro-His-Ala-Arg-Ile-Lys-NH2 by sub-cutaneous injection and immediately tested for bacterial infection. A Staphylococcus aureus infection is confirmed, and antibiotics administered. After a few days of once daily administration of the peptide, markers of Systematic Inflammatory Response Syndrome (SIRS), including temperature, heart rate and breathing rate improve and kidney function returns to normal. The peptide is continued to be administered once daily to the patient until the patient is recovered.

### Example 11

### SA Inhibitor for Treatment of Sterile Inflammatory Response Following Trauma

An unconscious 32-year-old motorcyclist is admitted to the ER suffering from severe trauma including multiple fractures and contusions following a road traffic accident He is no responsive to vocal or physical stimulation and a CT scan reveals a traumatic brain injury. The patient is admitted to an intensive care unit and placed in an induced coma. On the third day of admission the patient showed elevated heart rate and temperature, characteristic of post traumatic sepsis. The patient is administered 4 mg/kg of a peptide with a sequence of Ac-Pro-Pro-Pro-His-Pro-His-Ala-Arg-Ile-Lys-NH₂ (SEQ ID NO: 2) by sub-cutaneous injection and immediately tested for bacterial infection. The patients' cultures fail to reveal a bacterial infection. After a few days of once daily administration of the peptide, markers of Systematic Inflammatory Response Syndrome (SIRS), including temperature, heart rate and breathing rate improve. The peptide is continued to be administered until SIRS markers have returned to normal.

### Example 12

### Standalone SA Inhibitor to Reduce Viral Load

A male patient, aged 71, is brought into the hospital suffering from respiratory flu like symptoms. A blood sample is taken from the patient, who is confirmed by PCR analysis of viral RNA to have a SARS-COV-2 infection. The patient is administered 4 mg/kg of a peptide with a sequence of Ac-Pro-Pro-Pro-His-Pro-His-Ala-Arg-Ile-Lys-NH₂ (SEQ ID NO: 2) once daily by sub-cutaneous injection. After a few days, the viral load as determined by PCR, is lower than what would have been expected in the absence of the administration of the peptide. Administration of the peptide continues until the patient is confirmed to be virus free by PCR.

### Example 13

### Standalone SA Inhibitor to Reduce Viral Load and NETosis

A male patient, age 81, with a comorbidity of cardiovascular disease and diabetes is seen in the Emergency Room. The patient is running a temperature of 101° F. and is suffering from ARDS and flu like symptoms. PCR testing confirms the presence of a COVID-19 virus infection. The patient is administered 4 mg/kg of a peptide with a sequence of Ac-Pro-Pro-Pro-His-Pro-His-Ala-Arg-Ile-Lys-NH₂ (SEQ ID NO: 2) once daily by sub-cutaneous injection. After a few days of administering the peptide, the viral load, as measured by PCR, is reduced and lower than would be expected in the absence of the peptide. Additionally, the patient is found to have improved lung function with a higher measured SpO2 level resulting in a lower level of respiratory distress. Reduction of viral load persists for the term of the treatment and treatment is continued until the patient is confirmed to be virus free by PCR. Administration of the peptide is continued until the patient is able to maintain a normal SpO2 levels for seven days and is found to have a clear chest X-ray.

### Example 14

### SA Inhibitor and Therapeutic to Reduce NETosis

A 53 year-old male patient is examined by his physician and is found to have Acute Respiratory Distress Syndrome (ARDS). The patent is administered 4 mg/kg of a peptide with the sequence of Ac-Pro-Pro-Pro-His-Pro-His-Ala-Arg-Ile-Lys-NH₂ (SEQ ID NO: 2) (once daily by sub-cutaneous injection). The patient is also administered a DNase I through inhalation. After a few days of the peptide and DNase I administered to the patient, the patient is found to have improved lung function with higher measured SpO2 levels. Further, the patient is found to have a reduction in the ARDS. Administration of the peptide and DNase I is continued until the patient is able to maintain normal SpO2 levels for seven days with a clear chest X-ray.

### Example 15

### TKI and Oral Niacin Sustained Release for Reduction of Viral Load and NETosis

An 81 year-old female patient living in an assisted living facility is confirmed to be suffering from a SARS-COV-2 infection with pneumonia. The patient is administered 4 mg/kg of a peptide with the sequence of Ac-Pro-Pro-Pro-His-Pro-His-Ala-Arg-Ile-Lys-NH₂ (SEQ ID NO: 2) once daily by sub-cutaneous injection. The patient is also administered 375 mg Nispan® orally once nightly for 7 days followed by 500 mg Niacin® once nightly for 7 days and 750 mg Niaspan® (2×375 mg) for 7 days. After a few days, the viral load as determined by PCR is reduced as to what would be expected without treatment. Additionally, the patient shows improved lung function with higher measured SpO2 levels which result in a lower level of respiratory distress than if the treatment had not been administered. Reduction of viral load persists as long as the treatment continues until the patient is confirmed to be virus free by PCR. However, the patient continues to suffer from respiratory distress. The patient continues to be provided Niaspan® until the patient is able to maintain normal SpO2 levels for 7 days with a clear chest Xray.

### Example 16

### SA Inhibitor and Higher Dose Oral Niacin Sustained Release if Receiving CPAP and Confirmed COVID

A 33 year-old male patient, who is morbidly obese and suffers from diabetes is confirmed by PCR to be suffering from a SARS-COV-2 infection along with ARDS. The patient is also receiving Continuous Positive Airway Pressure (CPAP) support. The patient is administered 4 mg/kg of a peptide with the sequence of Ac-Pro-Pro-Pro-His-Pro-His-Ala-Arg-Ile-Lys-NH₂ (SEQ ID NO: 2) once daily by sub-cutaneous injection. The patient is also administered 750 mg Niaspan® (2×375 mg) orally once nightly for seven days followed by 1000 mg (2×500 mg) Niacin® once nightly for the next seven days and 1500 mg (3×500 mg) once nightly for the following seven days. 17 days following initiation of the treatment, the viral load was determined by PCR to be significantly reduced as compared to what would have been expected in the absence of the treatment. Additionally, the individual shows improved lung function with higher measured SpO2 levels resulting in a lower level of respiratory distress. As a result, the patient is able to discontinue the use of the CPAP. Such discontinuation would not likely have occurred if the Niaspan® had not been administered. Reduction of viral load persists and the treatment is discontinued once the patient is confirmed to be virus free by PCR. Reduction of respiratory distress persists providing the Niaspan® treatment is continued until the individual is able to maintain normal SpO2 levels for 7 days with a clear chest X-ray.

### Example 17

### SA Inhibitor and Higher Dose Oral Niacin Sustained Release Just ARDS

A 70 year-old female patient arrives in the Emergency Room. The physician seeing the patient determines the patient is suffering from ARDS. The patient is administered 4 mg/kg of a peptide with the sequence of Ac-Pro-Pro-Pro-His-Pro-His-Ala-Arg-Ile-Lys-NH₂ (SEQ ID NO: 2) once daily by sub-cutaneous injection. The patient is also administered 750 mg Niaspan® (2×375 mg) orally once nightly for seven days, followed by 1000 mg (2×500 mg) Niacin® once nightly for the following seven days and 1500 mg (3×500 mg) once nightly for an additional seven days. The patient shows improved lung function with higher measured SpO2 levels resulting in a lower level of respiratory distress. Reduction of respiratory distress persists so the Niaspan® treatment is continued until the individual is able to maintain normal SpO2 levels for seven days with a clear chest X-ray.

### Example 18

SA Inhibitor and Pulmozyme if ARDS i.e. Too Late for Oral Niacin

A 91 year-old male patient is admitted to the hospital and is found to have a COVID-19 infection by PCR. The patient is also suffering from ARDS. The patient is administered 4 mg/kg of a peptide with the sequence of Ac-Pro-Pro-Pro-His-Pro-His-Ala-Arg-Ile-Lys-NH₂ (SEQ ID NO: 2) once daily by sub-cutaneous injection. The patient is also administered Pulmozyme® (single use ampule) twice daily by nebulizer. After a few days the viral load as determined by PCR, is lower than would be observed in the absence of the treatment. Additionally, the individual shows improved lung function and higher measured SpO2 levels resulting in a lower level of respiratory distress. Reduction of viral load persists and the treatment is continued until the patient is confirmed to be virus free by PCR. Respiratory distress persists and the treatment is continued with only Pulmozyme® until the individual is able to maintain normal SpO2 levels for 7 days with a clear chest X-ray.

### Example 19

### SA Inhibitor, Niacin and Pulmozyme for COVID Induced ARDS

A 72 year-old female patient in an assisted care facility is tested by PCR and found to have a SARS-COV-2 infection and ARDS. The patient is administered 4 mg/kg of a peptide with the sequence Ac-Pro-Pro-Pro-His-Pro-His-Ala-Arg-Ile-Lys-NH₂ (SEQ ID NO: 2) once daily by sub-cutaneous injection. The patient was also administered 750 mg Niaspan® (2×375 mg) orally once nightly for seven days followed by 1000 mg (2×500 mg) Niacin® once nightly for an additional seven days and 1500 mg (3×500 mg) once nightly for a further seven days. The patient is further administered Pulmozyme® (single use ampule) twice daily by nebulizer. After a few days the viral load as determined by PCR, is reduced to a level below that which would be expected in the absence of the peptide. Additionally, the patient shows improved lung function and higher measured SpO2 levels resulting in a lower level of respiratory distress. Reduction of viral load persists as long as the treatment continues until the patient is confirmed to be virus free by PCR. Reduction of respiratory distress persists and the patient is continued to be administered the Niaspan®/Pulmozyme® treatment continues until the individual is able to maintain normal SpO2 levels for seven days with a clear chest X-ray.

### Example 20

### Combination Anti-Viral and SA Inhibitor to Reduce Viral Load and NETosis

A 68 year-old male enters the emergency room. The patient is tested and found by PCR to have a SARS-COV-2 infection. The patient is also found to require supplemental oxygen. The patient is administered 4 mg/kg of a peptide with the sequence Ac-Pro-Pro-Pro-His-Pro-His-Ala-Arg-Ile-Lys-NH₂ (SEQ ID NO: 2) once daily by intravenous injection. The patient is also administered Remdesivir by intravenous injection (200 mg on day 1 followed by 100 mg on days 2-10). After ten days the viral load, as determined by PCR, is lower than would be expected in the absence of treatment. Additionally, the patient shows improved lung function with higher measured SpO2 levels resulting in a lower level of respiratory distress. Reduction of viral load persists as long as the treatment continues until the patient is confirmed to be virus free by PCR test. Reduction of respiratory distress persists and the treatment is continued until the individual is able to maintain normal SpO2 levels for 7 days with a clear chest X-ray.

### Example 14

### SA Inhibitor and Therapeutic to Reduce NETosis

In this example, a 51 year-old male presents to his general practitioner suffering from high temperature, nausea, joint pains and red patches on his legs. The physician diagnoses a flare up of a pre-diagnosed Chron's disease and prescribes a dry powder inhaler formulation of a peptide with the sequence Ac-Pro-Pro-Pro-His-Pro-His-Ala-Arg-Ile-Lys-NH₂ (SEQ ID NO: 2). The patient is advised to take one metered dose (1 mg/kg) each morning until symptoms abate. After eight days the male goes back into remission and discontinues treatment.

TABLE 5
Sequence Listings

| SEQ ID NO: | Sequence |
|---|---|
| 1 | Pₙ-Xᵣ-H-P-H-A-R-I-K |

n is an integer from 1-10, X is any amino acid,
r is an integer from 0 to 2, m is an integer from
1 to 3,

| SEQ ID NO: | Sequence |
|---|---|
| 2 | P-P-P-H-P-H-A-R-I-K |

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

1. A pharmaceutical composition comprising one or more therapeutics to prevent or reduce the severity of an infection,
wherein, the one or more therapeutics is selected from a peptide that inhibits or prevents an interaction between a Src family kinase and an androgen receptor, a Niacin or Niacin derivative and/or a DNase I or DNase I derivative.

2. The pharmaceutical composition of claim 1, wherein the infection is a respiratory infection.

3. The pharmaceutical composition of claim 1, wherein the infection is caused by a virus, a bacteria or a fungus.

4. (canceled)

5. (canceled)

6. (canceled)

7. The pharmaceutical composition of claim 2, wherein the respiratory infection results in Acute Respiratory Distress Syndrome (ARDS).

8. The pharmaceutical composition of claim 1, wherein the one or more therapeutics comprises a peptide of SEQ ID NO: 1 or SEQ ID NO: 2.

9. (canceled)

10. (canceled)

11. The pharmaceutical composition of claim 1, where the one or more therapeutics comprises Niacin, a Niacin derivative or a Niacin precursor.

12. The pharmaceutical composition of claim 11, wherein the Niacin, Niacin derivative or Niacin precursor is selected from the group of nicotinic acid (pyridine carboxylic acid), nicotinamide (Niacinamide or pyridine-3-carboxamide) or nicotinamide riboside (1-(beta-D-Ribofuranosyl) nicotinamide.

13. (canceled)

14. The pharmaceutical composition of claim 1, wherein the one or more therapeutics comprises DNase I or a DNase I derivative that is human-derived or synthesized recombinantly.

15. (canceled)

16. (canceled)

17. A pharmaceutical composition comprising one or more therapeutics to prevent or reduce NETopathy in a subject,
wherein, the one or more therapeutics comprises a peptide that inhibits or prevents an interaction between a Src family kinase and an androgen receptor.

18. The pharmaceutical composition of claim 17, wherein the NETopathy is caused by an autoimmune disease.

19. The pharmaceutical composition of claim 18, wherein the autoimmune disease is selected from Addison's disease, celiac disease, dermatomyositis, fibromyalgia, Graves' disease, Guillain-Barre syndrome, Hashimoto thyroiditis, Kawasaki disease, multiple sclerosis, myasthenia gravis, pernicious anemia, psoriasis, reactive arthritis, rheumatic fever, rheumatoid arthritis, scleroderma, Sjögren syndrome, Systemic lupus erythematosus, type 1 diabetes, ulcerative colitis and vitiligo, COPD, Fibrosis, small vessel vasculitis, preeclampsia, endometriosis, psoriasis, gout, inflammatory bowel disease, Chron's disease, anti-phospholipid syndrome, sickle cell disease, graft vs host disease, organ transplant rejection, atherosclerosis, reperfusion injury, transfusion related lung injury (TRALI), type 2 diabetes, obesity, Alzheimer's Disease and gout.

20. The pharmaceutical composition of claim 17, wherein the NETopathy is caused by one of a viral infection, a bacterial infection, a fungal infection, alzheimer's disease, rheumatoid arthritis, vasculitis, preeclampsia, graft vs host disease, transplant rejection or trauma.

21. (canceled)

22. (canceled)

23. The pharmaceutical composition of claim 17, wherein the pharmaceutical composition further comprises a Niacin or Niacin derivative and/or a DNase I or DNase I derivative.

24. (canceled)

25. The pharmaceutical composition of claim 17, wherein the pharmaceutical composition further comprises a Niacin or Niacin derivative and/or a DNase I or DNase I derivative.

26. A pharmaceutical composition comprising one or more therapeutics to prevent or reduce the severity of an infection, the pharmaceutical composition comprised of a peptide with a SEQ ID NO: 2.

27. The pharmaceutical composition of claim 26, further comprising a Niacin or Niacin derivative and/or a DNase I or DNase I derivative.

28. The pharmaceutical composition of claim 26, wherein the peptide inhibits or prevents an interaction between a Src family kinase and an androgen receptor.

29. The pharmaceutical composition of claim 26, wherein the infection is a viral infection, bacterial infection or fungal infection.

30. (canceled)

31. The pharmaceutical composition of claim 26, wherein the infection is bacterial meningitis or bacterial sepsis.

32. (canceled)

33. The pharmaceutical composition of claim 26, wherein the infection is viral meningitis, influenza or pneumonia.

34. (canceled)

35. (canceled)

36. (canceled)
